SEQUENCE LISTING (15 sequences)

SEQ ID NO: 1 - protein, 967 aa, Arabidopsis thaliana
MANRKLEKMASIDVHLRQLVPGKVSEDDKLVEYDALLLDRFLDILQDLHGEDLRETVQELYEHSAEYEGKHEPKKLEELGSVLTSLDPGDSIVIAKAFSHMLNLANLAEEVQIAYRRRIKKLKKGDFVDESSATTESDLEETFKKLVGDLNKSPEEIFDALKNQTVDLVLTAHPTQSVRRSLLQKHGRIRDCLAQLYAKDITPDDKQELDEALQREIQAAFRTDEIKRTPPTPQDEMRAGMSYFHETIWKGVPKFLRRVDTALKNIGIEERVPYNAPLIQFSSWMGGDRDGNPRVTPEVTRDVCLLARMMAATMYFNQIEDLMFEMSMWRCNDELRARADEVHANSRKDAAKHYIEFWKSIPTTEPYRVILGDVRDKLYHTRERAHQLLSNGHSDVPVEATFINLEQFLEPLELCYRSLCSCGDRPIADGSLLDFLRQVSTFGLSLVRLDIRQESDRHTDVLDAITTHLDIGSYREWSEERRQEWLLSELSGKRPLFGSDLPKTEEIADVLDTFHVIAELPADSFGAYIISMATAPSDVLAVELLQRECRVKQPLRVVPLFEKLADLEAAPAAVARLFSVDWYKNRINGKQEVMIGYSDSGKDAGRLSAAWQLYKAQEELVKVAKEYGVKLTMFHGRGGTVGRGGGPTHLAILSQPPDTINGSLRVTVQGEVIEQSFGEEHLCFRTLQRFTAATLEHGMRPPISPKPEWRALLDEMAVVATEEYRSVVFQEPRFVEYFRLATPELEYGRMNIGSRPSKRKPSGGIESLRAIPWIFAWTQTRFHLPVWLGFGSAIRHVIEKDVRNLHMLQDMYQHWPFFRVTIDLIEMVFAKGDPGIAALYDKLLVSEELWPFGEKLRANFEETKKLILQTAGHKDLLEGDPYLKQRLRLRDSYITTLNVCQAYTLKRIRDPSYHVTLRPHISKEIAESSKPAKELIELNPTSEYAPGLEDTLILTMKGIAAGLQNTG

SEQ ID NO: 2 - protein, 960 aa, Zea mays
MPERHQSIDAQLRLLAPGKVSEDDKLVEYDALLVDRFLDILQDLHGPHLREFVQECYELSAEYENDRDEARLGELGSKLTSLPPGDSIVVASSFSHMLNLANLAEEVQIAHRRRIKLKRGDFADEASAPTESDIEETLKRLVSQLGKSREEVFDALKNQTVDLVFTAHPTQSVRRSLLQKHGRIRNCLRQLYAKDITADDKQELDEALQREIQAAFRTDEIRRTPPTPQDEMRAGMSYFHETIWKGVPKFLRRIDTALKNIGINERLPYNAPLIQFSSWMGGDRDGNPRVTPEVTRDVCLLARMMAANLYFSQIEDLMFELSMWRCSDELRIRADELHRSSRKAAKHYIEFWKQVPPNEPYRVILGDVRDKLYYTRERSRHLLTSGISEILEEATFTNVEQFLEPLELCYRSLCACGDKPIADGSLLDFLRQVSTFGLALVKLDIRQESDRHTDVLDSITTHLGIGSYAEWSEEKRQDWLLSELRGKRPLFGSDLPQTEETADVLGTFHVLAELPADCFGAYIISMATAPSDVLAVELLQRECHVKHPLRVVPLFEKLADLEAAPAAVARLFSIDWYMDRINGKQEVMIGYSDSGKDAGRLSAAWQMYKAQEELIKVAKHYGVKLTMFHGRGGTVGRGGGPTHLAILSQPPDTIHGSLRVTVQGEVIEHSFGEELLCFRTLQRYTAATLEHGMHPPISPKPEWRALMDEMAVVATKEYRSIVFQEPRFVEYFRSATPETEYGRMNIGSRPSKRKPSGGIESLRAIPWIFAWTQTRFHLPVWLGFGAAIKHIMQKDIRNIHILREMYNEWPFFRVTLDLLEMVFAKGDPGIAAVYDKLLVADDLQSFGEQLRKNYEETKELLLQVAGHKDVLEGDPYLKQRLRLRESYITTLNVCQAYTLKRIRDPSFQVSPQPPLSKEFTDESQPAELVQLNQQSEYAPGLEDTLILTMKGIAAGMQNTG

SEQ ID NO: 3 - protein, 966 aa, Glycine max
MGTRNFEKMASIDAQLRLLAPSKVSDDDKLVEYDALLLDRFLDILQDLHGDDIRETVQDCYELSAEYEGQNNPQKLEELGNMLTGLDAGDSIVISKSFAHMLNLANLAEEVQIAYRRRIKLLKKGDFADENSAITESDIEETFKRLVNQLKKTPQEIFDALKSQTVDLVLTAHPTQSVRRSLLQKHGRIRNCLTQLYAKDITPDDKQELDEALQREIQAAFRTDEIRRTPPTPQDEMRAGMSYFHETIWKGIPKFLRRVDTALKNIGINERVPYNAPVIQFSSWMGGDRDGNPRVTPEVTRDVCLLARMMAANLYFSQIEDLMFELSMWRCNDELRVRSDELLSSSKRDAKHYIEFWKQIPPNEPYRVILGDVRDKLYNTRERARQLLANGSSEIPEETTFTNVEQFLEPLELCYRSLCACGDQPIADGSLLDFLRQVSTFGLSLVRLDIRQESDRHTDVMDAITNHLEIGSYREWSEERRQEWLLSELSGKRPLFGPDLPKTEEIADVLETFHVIAELPSDSFGAYIISMATAPSDVLSVELLQRECHVKQPLRVVPLFEKLADLEAAPAAVARLFSIDWYRDRINGKQEVMIGYSDSGKDAGRFSAAWALYKAQEELIKVAKEFGVKLTMFHGRGGTVGRGGGPTHLAILSQPPDTIHGSLRVTVQGEVIEQSFGEEHLCFRTLQRFTAATLEHGMHPPVAPKPEWRALMDEMAVIATEEYRSIVFQEPRFVEYFRCATPELEYGRMNIGSRPSKRKPSGGIESLRAIPWIFAWTQTRFHLPVWLGFGAAFSHVIKKDPKNLQMLQDMYNQWPFFRVSLDLVEMVFAKGDPGIAALYDKLLVSEELWPFGERLRSMFEETKSLLLQVAGHKDLLEGDPYLKQRLRLRDSYITTLNVLQAYTLKRIRDPDYHVKLRPHLSKDYMESNKPAAELVKLNPTSDYAPGLEDTLILTMKGIAAGMQNTG

SEQ ID NO: 4 - protein, 972 aa, Triticum aestivum
MALSAPGGGSGKIERLSSIDAQLRLLVPAKVSEDDKLIEYDALLLDRFLDVLQGLHGDDLREMVQECYEVAAEYETKHDLEKLDELGEMITSLDPGDSIVIAKAFSHMLNLANLAEEVQIAYRRRVKLKKGDFADENSAITESDIEETLKRLVFDMKKSPAEVFDALKNQTVDLVLTAHPTQSVRRSLLQKHSRIRNCLVQLYSKDITPDDKQELDEALQREIQAAFRTDEIRRLSPTPQDHMRAGMSDFHETIWKGVPKFLRRVDTALKNIGINERVPYNAPLIQFSSWMGGDRDGNPRVTPEVTRDVCLLARMMAANLYCAQIEDLMFELSMWRCNDELRSRADELHRSSKKDAKHYIEFWKKVPPNEPYRVILGDVRDNLYNTRERSRELLSSGHSDIPEEATLTNLEQLLEPLELCYRSLCACGDRVIADGTLLDFLRQVSTFGLSLVKLDIRQESDRHTDALDAITSYLGIGSYREWSEEHRQEWLLSELNGKRPLFGADLPMTEEVADVMGAFQVIAELPGDNFGAYVISMATSPSDVLAVELLQRECHIKTPLRVVPLFEKLADLEAAPAALARLFSIDWYRERINGKQEVMIGYSDSGKDAGRLSAAWQMYKAQEDLVKVAKQFGVKLTMFHGRGGTVGRGGGPTHLAILSQPPDTINGSLRVTVQGEVIEQSFGEEHLCFRTLQRFTAATLEHGMRPPISPKPEWRALLDEMAVVATEEYRSIVFQEPRFVEYFRLATPETEYGRMNIGSRPSKRKPSGGIESLRAIPWIFAWTQTRFHLPVWLGFGGAFKHILKKDIRNFHMLQEMYNEWPFFRVTIDLVEMVFAKGNPGIAALYDRLLVSEGLQPLGEKLRANYEETQKLLLQVAGHKDLLEGDPYLKQRLRLRDAYITTMNVCQAYTLKRIRDPDYHVALRPHLSKEVMDTSKPAAELVTLNPASEYAPGLEDTLILTMKGIAAGLQNTG

SEQ ID NO: 5 - protein, 972 aa, Hordeum vulgare
MALSAPGGGSGKIERLSSIDAQLRLLVPAKVSEDDKLIEYDALLLDRFLDVLQGLHGDDLREMVQECYEVAAEYETKHDLEKLDELGEMITSLDPGDSIVIAKAFSHMLNLANLAEEVQIAYRRRVKLKKGDFADENSAITESDIEETLKRLVFDMKKSPAEVFDALKNQTVDLVLTAHPTQSVRRSLLQKHSRIRNCLVQLYSKDITPDDKQELDEALQREIQAAFRTDEIRRTQPTPQDEMRAGMSYFHETIWKGVPKFLRRVDTALKNIGINERVPYNAPLIQFSSWMGGDRDGNPRVTPEVTRDVCLLARMMAANLYCAQIEDLMFELSMWRCNDELRARADELHRSSKKDAKHYIEFWKKVPPNEPYRVILGDVRDNLYNTRERSRELLSSGHSDIPEEATLTNLEQLLEPLELCYRSLCACGDRVIADGTLLDFLRQVSTFGLSLVKLDIRQESDRHTDALDAITSYLGIGSYREWSEERRQEWLLSELNGKRPLFGADLPMTEEVADVMGAFQVIAELPGDNFGAYVISMATSPSDVLAVELLQRECHIKTPLRVVPLFEKLADLEAAPAALARLFSIDWYRERINGKQEVMIGYSDSGKDAGRLSAAWQMYKAQEDLVKVAKQFGVKLTMFHGRGGTVGRGGGPTHLAILSQPPDTINGSLRVTVQGEVIEQSFGEEHLCFRTLQRFTAATLEHGMRPPISPKPEWRALLDEMAVVATEEYRSIVFQEPRFVEYFRLATPETEYGRMNIGSRPSKRKPSGGIESLRAIPWIFAWTQTRFHLPVWLGFGGAFKHILKKDIRNFHMLQEMYNEWPFFRVTIDLVEMVFAKGNPGIAALYDRLLVSEGLQPLGEKLRANYEETQKLLLQVAGHKDLLEGDPYLKQRLRLRDAYITTMNVCQAYTLKRIRDPDYHVALRPHLSKEVMDTSKPAAELVTLNPASEYAPGLEDTLILTMKGIAAGLQNTG

SEQ ID NO: 6 - protein, 964 aa, Oryza sativa
MAGKVEKMASIDAQLRMLAPAKLSEDDKLVEYDALLLDRFLDILQDLHGDDLRELVQECYEIAAEYEGKHDSQKLDELGNMLTSLDPGDSIVMAKAFSHMLNLANLAEEVQIAYRRRIKLKKGDFADENSALTESDIEETFKRLVVDLKKSPAEVFDALKSQTVDLVLTAHPTQSVRRSLLQKHSRIRNCLVQLYSKDITPDDKQELDEALQREIQAAFRTDEIRRTQPTPQDEMRAGMSYFHETIWKGVPKFLRRLDTALKNIGIDERVPYNAPLIQFSSWMGGDRDGNPRVTPEVTRDVCLLARMMASNLYCSQIEDLMFELSMWRCNDELRARADELHLSSKKDAKHYIEFWKKVPPSEPYRVVLGDVRDKLYNTRERARQLLSSGYSDIPEETTLTSVEQFLEPLELCYRSLCDCGDRVIADGTLLDFLRQVSTFGLCLVRLDIRQESDRHTDVLDAITTYLGIGSYREWSEERRQDWLLSELNGKRPLFGPDLPKTDEIADVLDTFRVIAELPADNFGAYIISMATAPSDVLAVELLQRECHVKTPLRVVPLFEKLADLESAPAAVARLFSIDWYRERINGKQEVMIGYSDSGKDAGRLSAAWQLYKSQEELINVAKEFGVKLTMFHGRGGTVGRGGGPTHLAILSQPPDTIHGSLRVTVQGEVIEQSFGEEHLCFRTLQRFTAATLEHGMHPPIAPKPEWRALLDEMAVVATKEYRSIVFQEPRFVEYFRLATPEMEYGRMNIGSRPSKRKPSGGIESLRAIPWIFAWTQTRFHLPVWLGFGSAFKHILEKDIRNLHMLQEMYNEWPFFRVTIDLVEMVFAKGDPGIAALYDKLLVSEELWPLGEKLRANCEETKQLLLQVAGHKDLLEGDLYLKQRLRLRNAYITTLNVCQAYTMKRIRDPDYHVTLRPHMSKEIMDWSKPAAELVKLNPTSEYAPGLEDTLILTMKGIAAGMQNTG

SEQ ID NO: 7 - protein, 964 aa, Sorghum bicolor
MAGKLEKMASIDAQLRMLAPAKLSEDDKLVEYDALLLDRFLDILQDLHGEDLRELVQECYEIAAEYERKHDSEKLDELGNMLTSLDPGDSIVTAKAFSHMLNLANLAEEVQIAYRRRIKLKKGDFADENSALTESDIEETFKRLVVDLKKSPAEVFDALKSQTVDLVLTAHPTQSVRRSLLQKHSRIRNCLVQLCSKDITPDDKQELDEALQREIQAAFRTDEIRRTQPTPQDEMRAGMSYFHETIWKGVPKFLRRVDTALKNIGIDERVPYNAPLIQFSSWMGGDRDGNPRVTPEVTRDVCLLARMMAANLYCSQIENLMFELSMWRCNDELRAQADELHRSSKKDAKHYIEFWKKVPPSEPYRVILGDLRDKLYNTRERARQLLSSGYSDIPEESTVTNVEQFLEPLELCYRSLCACGDRVIADGSLLDFLRQVSTFGLCLVRLDIRQESDRHTDVLDAITTYLGIGSYREWSEERRQEWLLSELNGKRPLFGPDLPTTDEIADVLDTFRVIAELPADNFGAYIISMATAPSDVLAVELLQRECHVKTPLRVVPLFEKLADLEGAPAALARLFSVDWYRERINGKQEVMIGYSDSGKDAGRLSAAWQLYKAQEELIKVAKKFGVKLTMFHGRGGTVGRGGGPTHLAILSQPPDTIHGSLRVTVQGEVIEQSFGEEHLCFRTLQRFTAATLEHGMHPPISPKPEWRALLDEMAVVATKEYRSIVFQEPRFVEYFRLATPEMEYGRMNIGSRPSKRKPSGGIESLRAIPWIFAWTQTRFHLPVWLGFGAAFKHILEKDIRNLHMLQEMYNEWPFFRVTIDLVEMVFAKGDPGIAALYDKLLVSSELWPLGEKLRANYEETKRLLLQVAGHKDLLEGDLYLKQRLRLRDAYITTLNVCQAYTMKRIRDPDYHVTLRPHLSKEIMDWNKPAAELVKLNPTSEYAPGLEDTLILTMKGIAAGMQNTG

SEQ ID NO: 8 - protein, 967 aa, synthetic construct
  REGION 1..967: note = Synthetic Construct
  VARIANT 1: note = Xaa = Any Amino Acid and can be present or absent
  VARIANT 2: note = Xaa = Any Amino Acid and can be present or absent
  VARIANT 3: note = Xaa = Any Amino Acid and can be present or absent
  VARIANT 4: note = Xaa = Any Amino Acid and can be present or absent
  VARIANT 5: note = Xaa = Any Amino Acid
  VARIANT 6: note = Xaa = Any Amino Acid
  VARIANT 8: note = Xaa = Any Amino Acid
  VARIANT 9: note = Xaa = Any Amino Acid
  VARIANT 10: note = Xaa = Any Amino Acid
  VARIANT 14: note = Xaa = Any Amino Acid
  VARIANT 15: note = Xaa = Any Amino Acid
  VARIANT 18: note = Xaa = Any Amino Acid
  VARIANT 20: note = Xaa = Any Amino Acid
  VARIANT 22: note = Xaa = Any Amino Acid
  VARIANT 24: note = Xaa = Any Amino Acid
  VARIANT 26: note = Xaa = Any Amino Acid
  VARIANT 31: note = Xaa = Any Amino Acid
  VARIANT 38: note = Xaa = Any Amino Acid
  VARIANT 44: note = Xaa = Any Amino Acid
  VARIANT 47: note = Xaa = Any Amino Acid
  VARIANT 51: note = Xaa = Any Amino Acid
  VARIANT 52: note = Xaa = Any Amino Acid
  VARIANT 53: note = Xaa = Any Amino Acid
  VARIANT 56: note = Xaa = Any Amino Acid
  VARIANT 59: note = Xaa = Any Amino Acid
  VARIANT 60: note = Xaa = Any Amino Acid
  VARIANT 63: note = Xaa = Any Amino Acid
  VARIANT 64: note = Xaa = Any Amino Acid
  VARIANT 69: note = Xaa = Any Amino Acid
  VARIANT 70: note = Xaa = Any Amino Acid
  VARIANT 71: note = Xaa = Any Amino Acid
  VARIANT 72: note = Xaa = Any Amino Acid
  VARIANT 73: note = Xaa = Any Amino Acid
  VARIANT 74: note = Xaa = Any Amino Acid
  VARIANT 75: note = Xaa = Any Amino Acid
  VARIANT 77: note = Xaa = Any Amino Acid
  VARIANT 81: note = Xaa = Any Amino Acid
  VARIANT 82: note = Xaa = Any Amino Acid
  VARIANT 83: note = Xaa = Any Amino Acid
  VARIANT 85: note = Xaa = Any Amino Acid
  VARIANT 87: note = Xaa = Any Amino Acid
  VARIANT 88: note = Xaa = Any Amino Acid
  VARIANT 94: note = Xaa = Any Amino Acid
  VARIANT 95: note = Xaa = Any Amino Acid
  VARIANT 96: note = Xaa = Any Amino Acid
  VARIANT 97: note = Xaa = Any Amino Acid
  VARIANT 99: note = Xaa = Any Amino Acid
  VARIANT 115: note = Xaa = Any Amino Acid
  VARIANT 122: note = Xaa = Any Amino Acid
  VARIANT 124: note = Xaa = Any Amino Acid
  VARIANT 128: note = Xaa = Any Amino Acid
  VARIANT 131: note = Xaa = Any Amino Acid
  VARIANT 134: note = Xaa = Any Amino Acid
  VARIANT 139: note = Xaa = Any Amino Acid
  VARIANT 143: note = Xaa = Any Amino Acid
  VARIANT 145: note = Xaa = Any Amino Acid
  VARIANT 148: note = Xaa = Any Amino Acid
  VARIANT 149: note = Xaa = Any Amino Acid
  VARIANT 150: note = Xaa = Any Amino Acid
  VARIANT 151: note = Xaa = Any Amino Acid
  VARIANT 153: note = Xaa = Any Amino Acid
  VARIANT 154: note = Xaa = Any Amino Acid
  VARIANT 155: note = Xaa = Any Amino Acid
  VARIANT 157: note = Xaa = Any Amino Acid
  VARIANT 163: note = Xaa = Any Amino Acid
  VARIANT 187: note = Xaa = Any Amino Acid
  VARIANT 191: note = Xaa = Any Amino Acid
  VARIANT 194: note = Xaa = Any Amino Acid
  VARIANT 197: note = Xaa = Any Amino Acid
  VARIANT 198: note = Xaa = Any Amino Acid
  VARIANT 203: note = Xaa = Any Amino Acid
  VARIANT 227: note = Xaa = Any Amino Acid
  VARIANT 229: note = Xaa = Any Amino Acid
  VARIANT 230: note = Xaa = Any Amino Acid
  VARIANT 236: note = Xaa = Any Amino Acid
  VARIANT 243: note = Xaa = Any Amino Acid
  VARIANT 252: note = Xaa = Any Amino Acid
  VARIANT 259: note = Xaa = Any Amino Acid
  VARIANT 269: note = Xaa = Any Amino Acid
  VARIANT 272: note = Xaa = Any Amino Acid
  VARIANT 278: note = Xaa = Any Amino Acid
  VARIANT 312: note = Xaa = Any Amino Acid
  VARIANT 313: note = Xaa = Any Amino Acid
  VARIANT 314: note = Xaa = Any Amino Acid
  VARIANT 316: note = Xaa = Any Amino Acid
  VARIANT 317: note = Xaa = Any Amino Acid
  VARIANT 321: note = Xaa = Any Amino Acid
  VARIANT 326: note = Xaa = Any Amino Acid
  VARIANT 332: note = Xaa = Any Amino Acid
  VARIANT 337: note = Xaa = Any Amino Acid
  VARIANT 338: note = Xaa = Any Amino Acid
  VARIANT 339: note = Xaa = Any Amino Acid
  VARIANT 342: note = Xaa = Any Amino Acid
  VARIANT 343: note = Xaa = Any Amino Acid
  VARIANT 344: note = Xaa = Any Amino Acid
  VARIANT 345: note = Xaa = Any Amino Acid
  VARIANT 347: note = Xaa = Any Amino Acid
  VARIANT 348: note = Xaa = Any Amino Acid
  VARIANT 349: note = Xaa = Any Amino Acid
  VARIANT 350: note = Xaa = Any Amino Acid
  VARIANT 360: note = Xaa = Any Amino Acid
  VARIANT 361: note = Xaa = Any Amino Acid
  VARIANT 363: note = Xaa = Any Amino Acid
  VARIANT 364: note = Xaa = Any Amino Acid
  VARIANT 370: note = Xaa = Any Amino Acid
  VARIANT 374: note = Xaa = Any Amino Acid
  VARIANT 377: note = Xaa = Any Amino Acid
  VARIANT 380: note = Xaa = Any Amino Acid
  VARIANT 385: note = Xaa = Any Amino Acid
  VARIANT 386: note = Xaa = Any Amino Acid
  VARIANT 387: note = Xaa = Any Amino Acid
  VARIANT 390: note = Xaa = Any Amino Acid
  VARIANT 391: note = Xaa = Any Amino Acid
  VARIANT 393: note = Xaa = Any Amino Acid
  VARIANT 395: note = Xaa = Any Amino Acid
  VARIANT 396: note = Xaa = Any Amino Acid
  VARIANT 397: note = Xaa = Any Amino Acid
  VARIANT 398: note = Xaa = Any Amino Acid
  VARIANT 400: note = Xaa = Any Amino Acid
  VARIANT 402: note = Xaa = Any Amino Acid
  VARIANT 403: note = Xaa = Any Amino Acid
  VARIANT 404: note = Xaa = Any Amino Acid
  VARIANT 405: note = Xaa = Any Amino Acid
  VARIANT 408: note = Xaa = Any Amino Acid
  VARIANT 421: note = Xaa = Any Amino Acid
  VARIANT 425: note = Xaa = Any Amino Acid
  VARIANT 426: note = Xaa = Any Amino Acid
  VARIANT 431: note = Xaa = Any Amino Acid
  VARIANT 445: note = Xaa = Any Amino Acid
  VARIANT 448: note = Xaa = Any Amino Acid
  VARIANT 461: note = Xaa = Any Amino Acid
  VARIANT 462: note = Xaa = Any Amino Acid
  VARIANT 464: note = Xaa = Any Amino Acid
  VARIANT 467: note = Xaa = Any Amino Acid
  VARIANT 468: note = Xaa = Any Amino Acid
  VARIANT 470: note = Xaa = Any Amino Acid
  VARIANT 475: note = Xaa = Any Amino Acid
  VARIANT 481: note = Xaa = Any Amino Acid
  VARIANT 484: note = Xaa = Any Amino Acid
  VARIANT 491: note = Xaa = Any Amino Acid
  VARIANT 499: note = Xaa = Any Amino Acid
  VARIANT 503: note = Xaa = Any Amino Acid
  VARIANT 505: note = Xaa = Any Amino Acid
  VARIANT 507: note = Xaa = Any Amino Acid
  VARIANT 511: note = Xaa = Any Amino Acid
  VARIANT 512: note = Xaa = Any Amino Acid
  VARIANT 513: note = Xaa = Any Amino Acid
  VARIANT 515: note = Xaa = Any Amino Acid
  VARIANT 517: note = Xaa = Any Amino Acid
  VARIANT 522: note = Xaa = Any Amino Acid
  VARIANT 524: note = Xaa = Any Amino Acid
  VARIANT 529: note = Xaa = Any Amino Acid
  VARIANT 535: note = Xaa = Any Amino Acid
  VARIANT 541: note = Xaa = Any Amino Acid
  VARIANT 550: note = Xaa = Any Amino Acid
  VARIANT 551: note = Xaa = Any Amino Acid
  VARIANT 553: note = Xaa = Any Amino Acid
  VARIANT 569: note = Xaa = Any Amino Acid
  VARIANT 574: note = Xaa = Any Amino Acid
  VARIANT 580: note = Xaa = Any Amino Acid
  VARIANT 584: note = Xaa = Any Amino Acid
  VARIANT 585: note = Xaa = Any Amino Acid
  VARIANT 607: note = Xaa = Any Amino Acid
  VARIANT 612: note = Xaa = Any Amino Acid
  VARIANT 613: note = Xaa = Any Amino Acid
  VARIANT 616: note = Xaa = Any Amino Acid
  VARIANT 619: note = Xaa = Any Amino Acid
  VARIANT 621: note = Xaa = Any Amino Acid
  VARIANT 622: note = Xaa = Any Amino Acid
  VARIANT 626: note = Xaa = Any Amino Acid
  VARIANT 627: note = Xaa = Any Amino Acid
  VARIANT 643: note = Xaa = Any Amino Acid
  VARIANT 661: note = Xaa = Any Amino Acid
  VARIANT 675: note = Xaa = Any Amino Acid
  VARIANT 681: note = Xaa = Any Amino Acid
  VARIANT 690: note = Xaa = Any Amino Acid
  VARIANT 700: note = Xaa = Any Amino Acid
  VARIANT 713: note = Xaa = Any Amino Acid
  VARIANT 719: note = Xaa = Any Amino Acid
  VARIANT 722: note = Xaa = Any Amino Acid
  VARIANT 727: note = Xaa = Any Amino Acid
  VARIANT 740: note = Xaa = Any Amino Acid
  VARIANT 745: note = Xaa = Any Amino Acid
  VARIANT 792: note = Xaa = Any Amino Acid
  VARIANT 794: note = Xaa = Any Amino Acid
  VARIANT 795: note = Xaa = Any Amino Acid
  VARIANT 797: note = Xaa = Any Amino Acid
  VARIANT 798: note = Xaa = Any Amino Acid
  VARIANT 799: note = Xaa = Any Amino Acid
  VARIANT 802: note = Xaa = Any Amino Acid
  VARIANT 803: note = Xaa = Any Amino Acid
  VARIANT 805: note = Xaa = Any Amino Acid
  VARIANT 806: note = Xaa = Any Amino Acid
  VARIANT 807: note = Xaa = Any Amino Acid
  VARIANT 809: note = Xaa = Any Amino Acid
  VARIANT 810: note = Xaa = Any Amino Acid
  VARIANT 813: note = Xaa = Any Amino Acid
  VARIANT 814: note = Xaa = Any Amino Acid
  VARIANT 821: note = Xaa = Any Amino Acid
  VARIANT 822: note = Xaa = Any Amino Acid
  VARIANT 825: note = Xaa = Any Amino Acid
  VARIANT 833: note = Xaa = Any Amino Acid
  VARIANT 839: note = Xaa = Any Amino Acid
  VARIANT 842: note = Xaa = Any Amino Acid
  VARIANT 846: note = Xaa = Any Amino Acid
  VARIANT 847: note = Xaa = Any Amino Acid
  VARIANT 848: note = Xaa = Any Amino Acid
  VARIANT 850: note = Xaa = Any Amino Acid
  VARIANT 851: note = Xaa = Any Amino Acid
  VARIANT 852: note = Xaa = Any Amino Acid
  VARIANT 855: note = Xaa = Any Amino Acid
  VARIANT 858: note = Xaa = Any Amino Acid
  VARIANT 859: note = Xaa = Any Amino Acid
  VARIANT 860: note = Xaa = Any Amino Acid
  VARIANT 864: note = Xaa = Any Amino Acid
  VARIANT 865: note = Xaa = Any Amino Acid
  VARIANT 867: note = Xaa = Any Amino Acid
  VARIANT 870: note = Xaa = Any Amino Acid
  VARIANT 876: note = Xaa = Any Amino Acid
  VARIANT 881: note = Xaa = Any Amino Acid
  VARIANT 891: note = Xaa = Any Amino Acid
  VARIANT 892: note = Xaa = Any Amino Acid
  VARIANT 897: note = Xaa = Any Amino Acid
  VARIANT 900: note = Xaa = Any Amino Acid
  VARIANT 905: note = Xaa = Any Amino Acid
  VARIANT 912: note = Xaa = Any Amino Acid
  VARIANT 913: note = Xaa = Any Amino Acid
  VARIANT 914: note = Xaa = Any Amino Acid
  VARIANT 916: note = Xaa = Any Amino Acid
  VARIANT 917: note = Xaa = Any Amino Acid
  VARIANT 918: note = Xaa = Any Amino Acid
  VARIANT 920: note = Xaa = Any Amino Acid
  VARIANT 921: note = Xaa = Any Amino Acid
  VARIANT 924: note = Xaa = Any Amino Acid
  VARIANT 925: note = Xaa = Any Amino Acid
  VARIANT 926: note = Xaa = Any Amino Acid
  VARIANT 927: note = Xaa = Any Amino Acid
  VARIANT 928: note = Xaa = Any Amino Acid
  VARIANT 929: note = Xaa = Any Amino Acid
  VARIANT 930: note = Xaa = Any Amino Acid
  VARIANT 933: note = Xaa = Any Amino Acid
  VARIANT 936: note = Xaa = Any Amino Acid
  VARIANT 937: note = Xaa = Any Amino Acid
  VARIANT 940: note = Xaa = Any Amino Acid
  VARIANT 941: note = Xaa = Any Amino Acid
  VARIANT 943: note = Xaa = Any Amino Acid
  VARIANT 963: note = Xaa = Any Amino Acid
XXXXXXEXXXSIDXXLRXLXPXKXSXDDKLXEYDALLXDRFLDXLQXLHGXXXREXVQXXYEXXAEYEXXXXXXXLXELTXXXTXLXXGDSIVXXXXFXHMLNLANLAEEVQIAXRRRIKLXKXGDFXDEXSAXTESDXEEEXKXLVXXXXKXXXEXFDALKXQTVDLVLTAHPTQSVRRSLLQKHXRIRXCLXQLXXKDITXDDKQELDEALQREIQAAFRTDEIXRXXPTPQDXMRAGMSXFHETIWKGXPKFLRRXDTALKNIGIXERXPYNAPXIQFSSWMGGDRDGNPRVTPEVTRDVCLLARMMAXXXYXXQIEXLMFEXSMWRCXDELRXXXDEXXXXSXXXXAKHYIEFWKXXVXXEPYRVXLGDXRDXLYXTRERXXXLLXXGXSXXXXEXTXXXXEQXLEPLELCYRSLCXCGDXXIADGXLLDFLRQVSTFGLXLVXLDIRQESDRHTDXXDXITXXLXIGSYXEWSEEXRQXWLLSELXGKRPLFGXDLPXTXEXADVXXXFXVXAELPXDXFGAYXISMATXPSDVLXVELLQRECXXKXPLRVVPLFEKLADLEXAPAAXARLFSXDWYXXRINGKQEVMIGYSDSGKDAGRXSAAWXXYKXQEXLXXVAKXXGVKLTMFHGRGGTVGXGGGPTHLAILSQPPDTIXGSLRVTVQGEVIEXSFGEEXLCFRTLQRXTAATLEHGMXPPSSPKPEWRALXDEMAVXATXEYRSXVFQEPRFVEYFRXATPEXEYGRMNIGSRPSKRKPSGGIESLRAIPWIFAWTQTRFHLPVWLGFGXAXXHXXXKDXXNXXXLXXMYXXWPFFRVXXDLXEMVFAKGXPGIAAXYDXLLVXXXLXXXGEXLRXXXEETXXLXLQXAGHKDXLEGDXYLKQRLRLRXXYITTXNVXQAYTXKRIRDPXXXVXXXPXXSKXXXXXXXPAXELXXLNXXSXYAPGLEDTLILTMKGIAAGXQNTG

SEQ ID NO: 9 - protein, 967 aa, synthetic construct
  REGION 1..967: note = Synthetic Construct
  VARIANT 1: note = Xaa = Any Amino Acid and can be present or absent
  VARIANT 2: note = Xaa = Any Amino Acid and can be present or absent
  VARIANT 3: note = Xaa = Any Amino Acid and can be present or absent
  VARIANT 4: note = Xaa = Any Amino Acid and can be present or absent
  VARIANT 8: note = Xaa = K or R
  VARIANT 14: note = Xaa = A or V
  VARIANT 15: note = Xaa = Q or H
  VARIANT 20: note = Xaa = V or A
  VARIANT 24: note = Xaa = V or L
  VARIANT 26: note = Xaa = E or D
  VARIANT 31: note = Xaa = V or I
  VARIANT 38: note = Xaa = L or V
  VARIANT 44: note = Xaa = I or V
  VARIANT 47: note = Xaa = D or G
  VARIANT 52: note = Xaa = D or H
  VARIANT 53: note = Xaa = L or I
  VARIANT 59: note = Xaa = E or D
  VARIANT 60: note = Xaa = C or L
  VARIANT 64: note = Xaa = A or S
  VARIANT 75: note = Xaa = K or R
  VARIANT 83: note = Xaa = L or I
  VARIANT 85: note = Xaa = S or G
  VARIANT 87: note = Xaa = D or P
  VARIANT 88: note = Xaa = P or A
  VARIANT 95: note = Xaa = A or S
  VARIANT 96: note = Xaa = K or S
  VARIANT 97: note = Xaa = A or S
  VARIANT 99: note = Xaa = S or A
  VARIANT 115: note = Xaa = Y or H
  VARIANT 124: note = Xaa = K or R
  VARIANT 128: note = Xaa = A or V
  VARIANT 139: note = Xaa = I or L
  VARIANT 143: note = Xaa = F or L
  VARIANT 145: note = Xaa = R or K
  VARIANT 149: note = Xaa = D or Q
  VARIANT 150: note = Xaa = L or M
  VARIANT 153: note = Xaa = S or T
  VARIANT 154: note = Xaa = P or R
  VARIANT 157: note = Xaa = V or I
  VARIANT 163: note = Xaa = N or S
  VARIANT 170: note = Xaa = L or F
  VARIANT 187: note = Xaa = S or G
  VARIANT 197: note = Xaa = Y or C
  VARIANT 198: note = Xaa = S or A
  VARIANT 203: note = Xaa = P or A
  VARIANT 227: note = Xaa = R or K
  VARIANT 229: note = Xaa = T or L
  VARIANT 236: note = Xaa = E or H
  VARIANT 243: note = Xaa = Y or D
  VARIANT 252: note = Xaa = V or I
  VARIANT 259: note = Xaa = V or I
  VARIANT 269: note = Xaa = N or D
  VARIANT 278: note = Xaa = L or V
  VARIANT 312: note = Xaa = A or S
  VARIANT 313: note = Xaa = N or T
  VARIANT 314: note = Xaa = L or M
  VARIANT 316: note = Xaa = C or F
  VARIANT 321: note = Xaa = D or N
  VARIANT 326: note = Xaa = L or M
  VARIANT 332: note = Xaa = N or S
  VARIANT 338: note = Xaa = R or Q
  VARIANT 339: note = Xaa = A or S
  VARIANT 342: note = Xaa = L or V
  VARIANT 343: note = Xaa = H or L
  VARIANT 345: note = Xaa = S or N
  VARIANT 347: note = Xaa = K or R
  VARIANT 348: note = Xaa = K or R
  VARIANT 349: note = Xaa = D or A
  VARIANT 361: note = Xaa = V or I
  VARIANT 363: note = Xaa = P or T
  VARIANT 370: note = Xaa = I or V
  VARIANT 374: note = Xaa = V or L
  VARIANT 377: note = Xaa = K or N
  VARIANT 385: note = Xaa = A or S
  VARIANT 386: note = Xaa = R or H
  VARIANT 391: note = Xaa = S or N
  VARIANT 395: note = Xaa = D or E
  VARIANT 396: note = Xaa = I or V
  VARIANT 397: note = Xaa = P or L
  VARIANT 398: note = Xaa = E or V
  VARIANT 403: note = Xaa = T or I
  VARIANT 404: note = Xaa = N or S
  VARIANT 405: note = Xaa = V or L
  VARIANT 408: note = Xaa = F or L
  VARIANT 426: note = Xaa = P or V
  VARIANT 431: note = Xaa = S or T
  VARIANT 448: note = Xaa = R or K
  VARIANT 461: note = Xaa = V or A
  VARIANT 462: note = Xaa = L or M
  VARIANT 464: note = Xaa = S or A
  VARIANT 468: note = Xaa = Y or H
  VARIANT 475: note = Xaa = R or A
  VARIANT 484: note = Xaa = D or E
  VARIANT 505: note = Xaa = E or D
  VARIANT 511: note = Xaa = L or M
  VARIANT 513: note = Xaa = T or A
  VARIANT 517: note = Xaa = I or L
  VARIANT 529: note = Xaa = I or V
  VARIANT 535: note = Xaa = A or S
  VARIANT 541: note = Xaa = A or S
  VARIANT 550: note = Xaa = H or R
  VARIANT 551: note = Xaa = V or I
  VARIANT 569: note = Xaa = A or G
  VARIANT 574: note = Xaa = L or V
  VARIANT 580: note = Xaa = I or V
  VARIANT 607: note = Xaa = L or F
  VARIANT 612: note = Xaa = Q or A
  VARIANT 613: note = Xaa = L or M
  VARIANT 616: note = Xaa = S or A
  VARIANT 619: note = Xaa = E or D
  VARIANT 621: note = Xaa = I or V
  VARIANT 622: note = Xaa = K or N
  VARIANT 627: note = Xaa = F or Y
  VARIANT 661: note = Xaa = H or N
  VARIANT 675: note = Xaa = Q or H
  VARIANT 681: note = Xaa = H or L
  VARIANT 690: note = Xaa = F or Y
  VARIANT 703: note = Xaa = I or V
  VARIANT 704: note = Xaa = S or A
  VARIANT 713: note = Xaa = L or M
  VARIANT 719: note = Xaa = V or I
  VARIANT 722: note = Xaa = E or K
  VARIANT 727: note = Xaa = I or V
  VARIANT 794: note = Xaa = F or I
  VARIANT 797: note = Xaa = I or V
  VARIANT 803: note = Xaa = R or K
  VARIANT 806: note = Xaa = H or Q
  VARIANT 807: note = Xaa = I or M
  VARIANT 809: note = Xaa = Q or R
  VARIANT 810: note = Xaa = E or D
  VARIANT 813: note = Xaa = N or Q
  VARIANT 821: note = Xaa = T or S
  VARIANT 822: note = Xaa = I or L
  VARIANT 833: note = Xaa = D or N
  VARIANT 839: note = Xaa = L or V
  VARIANT 842: note = Xaa = K or R
  VARIANT 846: note = Xaa = S or A
  VARIANT 850: note = Xaa = W or Q
  VARIANT 851: note = Xaa = P or S
  VARIANT 852: note = Xaa = L or F
  VARIANT 859: note = Xaa = N or M
  VARIANT 864: note = Xaa = K or Q
  VARIANT 867: note = Xaa = L or I
  VARIANT 870: note = Xaa = V or T
  VARIANT 876: note = Xaa = L or D
  VARIANT 881: note = Xaa = P or L
  VARIANT 891: note = Xaa = D or E
  VARIANT 892: note = Xaa = A or S
  VARIANT 897: note = Xaa = L or M
  VARIANT 900: note = Xaa = C or L
  VARIANT 905: note = Xaa = L or M
  VARIANT 912: note = Xaa = D or S
  VARIANT 913: note = Xaa = Y or F
  VARIANT 914: note = Xaa = H or Q
  VARIANT 917: note = Xaa = L or P
  VARIANT 918: note = Xaa = R or Q
  VARIANT 920: note = Xaa = H or P
  VARIANT 924: note = Xaa = E or D
  VARIANT 927: note = Xaa = D or E
  VARIANT 929: note = Xaa = S or N
  VARIANT 930: note = Xaa = K or Q
  VARIANT 936: note = Xaa = V or I
  VARIANT 940: note = Xaa = P or Q
  VARIANT 943: note = Xaa = E or D
  VARIANT 963: note = Xaa = M or L
  VARIANT 5: note = Xaa = Any Amino Acid
  VARIANT 6: note = Xaa = Any Amino Acid
  VARIANT 9: note = Xaa = Any Amino Acid
  VARIANT 10: note = Xaa = Any Amino Acid
  VARIANT 18: note = Xaa = Any Amino Acid
  VARIANT 22: note = Xaa = Any Amino Acid
  VARIANT 51: note = Xaa = Any Amino Acid
  VARIANT 56: note = Xaa = Any Amino Acid
  VARIANT 63: note = Xaa = Any Amino Acid
  VARIANT 69: note = Xaa = Any Amino Acid
  VARIANT 70: note = Xaa = Any Amino Acid
  VARIANT 71: note = Xaa = Any Amino Acid
  VARIANT 72: note = Xaa = Any Amino Acid
  VARIANT 73: note = Xaa = Any Amino Acid
  VARIANT 74: note = Xaa = Any Amino Acid
  VARIANT 77: note = Xaa = Any Amino Acid
  VARIANT 81: note = Xaa = Any Amino Acid
  VARIANT 82: note = Xaa = Any Amino Acid
  VARIANT 94: note = Xaa = Any Amino Acid
  VARIANT 122: note = Xaa = Any Amino Acid
  VARIANT 131: note = Xaa = Any Amino Acid
  VARIANT 134: note = Xaa = Any Amino Acid
  VARIANT 148: note = Xaa = Any Amino Acid
  VARIANT 151: note = Xaa = Any Amino Acid
  VARIANT 155: note = Xaa = Any Amino Acid
  VARIANT 191: note = Xaa = Any Amino Acid
  VARIANT 194: note = Xaa = Any Amino Acid
  VARIANT 230: note = Xaa = Any Amino Acid
  VARIANT 272: note = Xaa = Any Amino Acid
  VARIANT 317: note = Xaa = Any Amino Acid
  VARIANT 337: note = Xaa = Any Amino Acid
  VARIANT 344: note = Xaa = Any Amino Acid
  VARIANT 350: note = Xaa = Any Amino Acid
  VARIANT 360: note = Xaa = Any Amino Acid
  VARIANT 364: note = Xaa = Any Amino Acid
  VARIANT 380: note = Xaa = Any Amino Acid
  VARIANT 387: note = Xaa = Any Amino Acid
  VARIANT 390: note = Xaa = Any Amino Acid
  VARIANT 393: note = Xaa = Any Amino Acid
  VARIANT 400: note = Xaa = Any Amino Acid
  VARIANT 402: note = Xaa = Any Amino Acid
  VARIANT 421: note = Xaa = Any Amino Acid
  VARIANT 425: note = Xaa = Any Amino Acid
  VARIANT 445: note = Xaa = Any Amino Acid
  VARIANT 467: note = Xaa = Any Amino Acid
  VARIANT 470: note = Xaa = Any Amino Acid
  VARIANT 481: note = Xaa = Any Amino Acid
  VARIANT 491: note = Xaa = Any Amino Acid
  VARIANT 499: note = Xaa = Any Amino Acid
  VARIANT 503: note = Xaa = Any Amino Acid
  VARIANT 507: note = Xaa = Any Amino Acid
  VARIANT 512: note = Xaa = Any Amino Acid
  VARIANT 515: note = Xaa = Any Amino Acid
  VARIANT 522: note = Xaa = Any Amino Acid
  VARIANT 524: note = Xaa = Any Amino Acid
  VARIANT 553: note = Xaa = Any Amino Acid
  VARIANT 584: note = Xaa = Any Amino Acid
  VARIANT 585: note = Xaa = Any Amino Acid
  VARIANT 626: note = Xaa = Any Amino Acid
  VARIANT 700: note = Xaa = Any Amino Acid
  VARIANT 740: note = Xaa = Any Amino Acid
  VARIANT 745: note = Xaa = Any Amino Acid
  VARIANT 792: note = Xaa = Any Amino Acid
  VARIANT 795: note = Xaa = Any Amino Acid
  VARIANT 798: note = Xaa = Any Amino Acid
  VARIANT 799: note = Xaa = Any Amino Acid
  VARIANT 802: note = Xaa = Any Amino Acid
  VARIANT 805: note = Xaa = Any Amino Acid
  VARIANT 814: note = Xaa = Any Amino Acid
  VARIANT 825: note = Xaa = Any Amino Acid
  VARIANT 847: note = Xaa = Any Amino Acid
  VARIANT 848: note = Xaa = Any Amino Acid
  VARIANT 855: note = Xaa = Any Amino Acid
  VARIANT 858: note = Xaa = Any Amino Acid
  VARIANT 860: note = Xaa = Any Amino Acid
  VARIANT 865: note = Xaa = Any Amino Acid
  VARIANT 916: note = Xaa = Any Amino Acid
  VARIANT 921: note = Xaa = Any Amino Acid
  VARIANT 925: note = Xaa = Any Amino Acid
  VARIANT 926: note = Xaa = Any Amino Acid
  VARIANT 928: note = Xaa = Any Amino Acid
  VARIANT 933: note = Xaa = Any Amino Acid
  VARIANT 937: note = Xaa = Any Amino Acid
  VARIANT 941: note = Xaa = Any Amino Acid
XXXXXXEXXXSIDXXLRXLXPXKXSXDDKLXEYDALLXDRFLDXLQXLHGXXXREXVQXXYEXXAEYEXXXXXXXLXELGXXXTXLXXGDSIVXXXXFXHMLNLANLAEEVQIAXRRRIKLXKXGDFXDEXSAXTESDXEETXKXLVXXXXKXXXEXFDALKXQTVDLVXTAHPTQSVRRSLLQKHXRIRXCLXQLXXKDITXDDKQELDEALQREIQAAFRTDEIXRXXPTPQDXMRAGMSXFHETIWKGXPKFLRRXDTALKNIGIXERXPYNAPXIQFSSWMGGDRDGNPRVTPEVTRDVCLLARMMAXXXYXXQIEXLMFEXSMWRCXDELRXXXDEXXXXSXXXXAKHYIEFWKXXPXXEPYRVXLGDXRDXLYXTRERXXXLLXXGXSXXXXEXTXXXXEQXLEPLELCYRSLCXCGDXXIADGXLLDFLRQVSTFGLXLVXLDIRQESDRHTDXXDXITXXLXIGSYXEWSEEXRQXWLLSELXGKRPLFGXDLPXTXEXADVXXXFXVXAELPXDXFGAYXISMATXPSDVLXVELLQRECXXKXPLRVVPLFEKLADLEXAPAAXARLFSXDWYXXRINGKQEVMIGYSDSGKDAGRXSAAWXXYKXQEXLXXVAKXXGVKLTMFHGRGGTVGRGGGPTHLAILSQPPDTIXGSLRVTVQGEVIEXSFGEEXLCFRTLQRXTAATLEHGMXPPXXPKPEWRALXDEMAVXATXEYRSXVFQEPRFVEYFRXATPEXEYGRMNIGSRPSKRKPSGGIESLRAIPWIFAWTQTRFHLPVWLGFGXAXXHXXXKDXXNXXXLXXMYXXWPFFRVXXDLXEMVFAKGXPGIAAXYDXLLVXXXLXXXGEXLRXXXEETXXLXLQXAGHKDXLEGDXYLKQRLRLRXXYITTXNVXQAYTXKRIRDPXXXVXXXPXXSKXXXXXXXPAXELXXLNXXSXYAPGLEDTLILTMKGIAAGXQNTG

SEQ ID NO: 10 - protein, 967 aa, synthetic construct
  REGION 1..967: note = Synthetic Construct
MANRKLEKMASIDVHLRQLVPGKVSEDDKLVEYDALLLDRFLDILQDLHGEDLRETVQELYEHSAEYEGKHEPKKLEELGSVLTSLDPGDSIVIAKAFSHMLNLANLAEEVQIAYRRRIKKLKKGDFVDESSATTESDLEETFKKLVGDLNKSPEEIFDALKNQTVDLVLTAHPTQSVRRSLLQKHGRIRDCLAQLYAKDITPDDKQELDEALQREIQAAFRTDEIKRTPPTPQDEMRAGMSYFHETIWKGVPKFLRRVDTALKNIGIEERVPYNAPLIQFSSWMGGDRDGNPRVTPEVTRDVCLLARMMAATMYFNQIEDLMFEMSMWRCNDELRARADEVHANSRKDAAKHYIEFWKSIPTTEPYRVILGDVRDKLYHTRERAHQLLSNGHSDVPVEATFINLEQFLEPLELCYRSLCSCGDRPIADGSLLDFLRQVSTFGLSLVRLDIRQESDRHTDVLDAITTHLDIGSYREWSEERRQEWLLSELSGKRPLFGSDLPKTEEIADVLDTFHVIAELPADSFGAYIISMATAPSDVLAVELLQRECRVKQPLRVVPLFEKLADLEAAPAAVARLFSVDWYKNRINGKQEVMIGYSDSGKDAGRLSAAWQLYKAQEELVKVAKEYGVKLTMFHGRGGTVGRGGGPTHLVILSQPPDTINGSLRVTVQGEVIEQSFGEEHLCFRTLQRFTAATLEHGMRPPISPKPEWRALLDEMAVVATEEYRSVVFQEPRFVEYFRLATPELEYGRMNIGSRPSKRKPSGGIESLRAIPWIFAWTQTRFHLPVWLGFGSAIRHVIEKDVRNLHMLQDMYQHWPFFRVTIDLIEMVFAKGDPGIAALYDKLLVSEELWPFGEKLRANFEETKKLILQTAGHKDLLEGDPYLKQRLRLRDSYITTLNVCQAYTLKRIRDPSYHVTLRPHISKEIAESSKPAKELIELNPTSEYAPGLEDTLILTMKGIAAGLQNTG

SEQ ID NO: 11 - protein, 967 aa, synthetic construct
  REGION 1..967: note = Synthetic Construct
MANRKLEKMASIDVHLRQLVPGKVSEDDKLVEYDALLLDRFLDILQDLHGEDLRETVQELYEHSAEYEGKHEPKKLEELGSVLTSLDPGDSIVIAKAFSHMLNLANLAEEVQIAYRRRIKKLKKGDFVDESSATTESDLEETFKKLVGDLNKSPEEIFDALKNQTVDLVLTAHPTQSVRRSLLQKHGRIRDCLAQLYAKDITPDDKQELDEALQREIQAAFRTDEIKRTPPTPQDEMRAGMSYFHETIWKGVPKFLRRVDTALKNIGIEERVPYNAPLIQFSSWMGGDRDGNPRVTPEVTRDVCLLARMMAATMYFNQIEDLMFEMSMWRCNDELRARADEVHANSRKDAAKHYIEFWKSIPTTEPYRVILGDVRDKLYHTRERAHQLLSNGHSDVPVEATFINLEQFLEPLELCYRSLCSCGDRPIADGSLLDFLRQVSTFGLSLVRLDIRQESDRHTDVLDAITTHLDIGSYREWSEERRQEWLLSELSGKRPLFGSDLPKTEEIADVLDTFHVIAELPADSFGAYIISMATAPSDVLAVELLQRECRVKQPLRVVPLFEKLADLEAAPAAVARLFSVDWYKNRINGKQEVMIGYSDSGKDAGRLSAAWQLYKAQEELVKVAKEYGVKLTMFHGRGGTVGRGGGPTHLAILSQPPDTINGSLRVTVQGEVIEQSFSEEHLCFRTLQRFTAATLEHGMRPPISPKPEWRALLDEMAVVATEEYRSVVFQEPRFVEYFRLATPELEYGRMNIGSRPSKRKPSGGIESLRAIPWIFAWTQTRFHLPVWLGFGSAIRHVIEKDVRNLHMLQDMYQHWPFFRVTIDLIEMVFAKGDPGIAALYDKLLVSEELWPFGEKLRANFEETKKLILQTAGHKDLLEGDPYLKQRLRLRDSYITTLNVCQAYTLKRIRDPSYHVTLRPHISKEIAESSKPAKELIELNPTSEYAPGLEDTLILTMKGIAAGLQNTG

SEQ ID NO: 12 - protein, 967 aa, synthetic construct
  REGION 1..967: note = Synthetic Construct
MANRKLEKMASIDVHLRQLVPGKVSEDDKLVEYDALLLDRFLDILQDLHGEDLRETVQELYEHSAEYEGKHEPKKLEELGSVLTSLDPGDSIVIAKAFSHMLNLANLAEEVQIAYRRRIKKLKKGDFVDESSATTESDLEETFKKLVGDLNKSPEEIFDALKNQTVDLVLTAHPTQSVRRSLLQKHGRIRDCLAQLYAKDITPDDKQELDEALQREIQAAFRTDEIKRTPPTPQDEMRAGMSYFHETIWKGVPKFLRRVDTALKNIGIEERVPYNAPLIQFSSWMGGDRDGNPRVTPEVTRDVCLLARMMAATMYFNQIEDLMFEMSMWRCNDELRARADEVHANSRKDAAKHYIEFWKSIPTTEPYRVILGDVRDKLYHTRERAHQLLSNGHSDVPVEATFINLEQFLEPLELCYRSLCSCGDRPIADGSLLDFLRQVSTFGLSLVRLDIRQESDRHTDVLDAITTHLDIGSYREWSEERRQEWLLSELSGKRPLFGSDLPKTEEIADVLDTFHVIAELPADSFGAYIISMATAPSDVLAVELLQRECRVKQPLRVVPLFEKLADLEAAPAAVARLFSVDWYKNRINGKQEVMIGYSDSGKDAGRLSAAWQLYKAQEELVKVAKEYGVKLTMFHGRGGTVGRGGGPTHLAILSQPPDTINGSLRVTVQGEVIEQSFGEEHLCFRTLQRFTAATLEHGMRPPISPKPEWRALLDEMAVVATEEYRSVVFQEPRFVEYFRLATPELEYGRMNIGSRPSKRKPSGGIESLRAIPWIFAWIQTRFHLPVWLGFGSAIRHVIEKDVRNLHMLQDMYQHWPFFRVTIDLIEMVFAKGDPGIAALYDKLLVSEELWPFGEKLRANFEETKKLILQTAGHKDLLEGDPYLKQRLRLRDSYITTLNVCQAYTLKRIRDPSYHVTLRPHISKEIAESSKPAKELIELNPTSEYAPGLEDTLILTMKGIAAGLQNTG

SEQ ID NO: 13 - protein, 967 aa, synthetic construct
  REGION 1..967: note = Synthetic Construct
MANRKLEKMASIDVHLRQLVPGKVSEDDKLVEYDALLLDRFLDILQDLHGEDLRETVQELYEHSAEYEGKHEPKKLEELGSVLTSLDPGDSIVIAKAFSHMLNLANLAEEVQIAYRRRIKKLKKGDFVDESSATTESDLEETFKKLVGDLNKSPEEIFDALKNQTVDLVLTAHPTQSVRRSLLQKHGRIRDCLAQLYAKDITPDDKQELDEALQREIQAAFRTDEIKRTPPTPQDEMRAGMSYFHETIWKGVPKFLRRVDTALKNIGIEERVPYNAPLIQFSSWMGGDRDGNPRVTPEVTRDVCLLARMMAATMYFNQIEDLMFEMSMWRCNDELRARADEVHANSRKDAAKHYIEFWKSIPTTEPYRVILGDVRDKLYHTRERAHQLLSNGHSDVPVEATFINLEQFLEPLELCYRSLCSCGDRPIADGSLLDFLRQVSTFGLSLVRLDIRQESDRHTDVLDAITTHLDIGSYREWSEERRQEWLLSELSGKRPLFGSDLPKTEEIADVLDTFHVIAELPADSFGAYIISMATAPSDVLAVELLQRECRVKQPLRVVPLFEKLADLEAAPAAVARLFSVDWYKNRINGKQEVMIGYSDSGKDAGRLSAAWQLYKAQEELVKVAKEYGVKLTMFHGRGGTVGRGGGPTHLVILSQPPDTINGSLRVTVQGEVIEQSFSEEHLCFRTLQRFTAATLEHGMRPPISPKPEWRALLDEMAVVATEEYRSVVFQEPRFVEYFRLATPELEYGRMNIGSRPSKRKPSGGIESLRAIPWIFAWTQTRFHLPVWLGFGSAIRHVIEKDVRNLHMLQDMYQHWPFFRVTIDLIEMVFAKGDPGIAALYDKLLVSEELWPFGEKLRANFEETKKLILQTAGHKDLLEGDPYLKQGLRLRDSYITTLNVCQAYTLKRIRDPSYHVTLRPHISKEIAESSKPAKELIELNPTSEYAPGLEDTLILTMKGIAAGLQNTG

SEQ ID NO: 14 - protein, 967 aa, synthetic construct
  REGION 1..967: note = Synthetic Construct
MANRKLEKMASIDVHLRQLVPGKVSEDDKLVEYDALLLDRFLDILQDLHGEDLRETVQELYEHSAEYEGKHEPKKLEELGSVLTSLDPGDSIVIAKAFSHMLNLANLAEEVQIAYRRRIKKLKKGDFVDESSATTESDLEETFKKLVGDLNKSPEEIFDALKNQTVDLVLTAHPTQSVRRSLLQKHGRIRDCLAQLYAKDITPDDKQELDEALQREIQAAFRTDEIKRTPPTPQDEMRAGMSYFHETIWKGVPKFLRRVDTALKNIGIEERVPYNAPLIQFSSWMGGDRDGNPRVTPEVTRDVCLLARMMAATMYFNQIEDLMFEMSMWRCNDELRARADEVHANSRKDAAKHYIEFWKSIPTTEPYRVILGDVRDKLYHTRERAHQLLSNGHSDVPVEATFINLEQFLEPLELCYRSLCSCGDRPIADGSLLDFLRQVSTFGLSLVRLDIRQESDRHTDVLDAITTHLDIGSYREWSEERRQEWLLSELSGKRPLFGSDLPKTEEIADVLDTFHVIAELPADSFGAYIISMATAPSDVLAVELLQRECRVKQPLRVVPLFEKLADLEAAPAAVARLFSVDWYKNRINGKQEVMIGYSDSGKDAGRLSAAWQLYKAQEELVKVAKEYGVKLTMFHGRGGTVGRGGGPTHLVILSQPPDTINGSLRVTVQGEVIEQSFSEEHLCFRTLQRFTAATLEHGMRPPISPKPEWRALLDEMAVVATEEYRSVVFQEPRFVEYFRLATPELEYGRMNIGSRPSKRKPSGGIESLRAIPWIFAWIQTRFHLPVWLGFGSAIRHVIEKDVRNLHMLQDMYQHWPFFRVTIDLIEMVFAKGDPGIAALYDKLLVSEELWPFGEKLRANFEETKKLILQTAGHKDLLEGDPYLKQGLRLRDSYITTLNVCQAYTLKRIRDPSYHVTLRPHISKEIAESSKPAKELIELNPTSEYAPGLEDTLILTMKGIAAGLQNTG

SEQ ID NO: 15 - protein, 970 aa, Zea mays
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIAHRRRNSKLKKGGFADEGSATTESDIEETLKRLVSEVGKSPEEVFEALKNQTVDLVFTAHPTQSARRSLLQKNARIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWPEDKRQEWLLSELRGKRPLLPPDLPQTDEIADVIGAFHVLAELPPDSFGPYIISMATAPSDVLAVELLQRECGVRQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEARFVEYFRSATPETEYGRMNIGSRPAKRRPGGGITTLRAIPWIFSWTQTRFHLPVWLGVGAAFKFAIDKDVRNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

(19) United States
(12) Patent Application Publication
(10) Pub. No.: US 2025/0197880 A1
(43) Pub. Date: Jun. 19, 2025

(54) ENGINEERED PHOSPHOENOLPYRUVATE CARBOXYLASE ENZYMES

(71) Applicant: The Regents of the University of California, Oakland, CA (US)

(72) Inventor: Paul B. LARSEN, Riverside, CA (US)

(73) Assignee: The Regents of the University of California, Oakland, CA (US)

(21) Appl. No.: 18/970,695

(22) Filed: Dec. 5, 2024

Related U.S. Application Data
(63) Continuation of application No. 17/601,913, filed on Oct. 6, 2021, now Pat. No. 12,168,773.
(60) Provisional application No. 62/832,727, filed on Apr. 11, 2019.

(51) Int. Cl. C12N 15/82; C12N 9/88

(52) U.S. Cl. CPC C12N 15/8271; C12N 9/88; C12N 15/8243; C12N 15/8262; C12Y 401/01031; C12N 2820/002; C12N 2820/007

(57) ABSTRACT

The present disclosure provides plants that express a variant phosphoenolpyruvate carboxylase (PEPC) enzyme. The plants have enhanced resistance to aluminum than comparable plants that lack the variant PEPC enzyme. In addition, the plants more effectively sequester carbon, extract phosphate, and produce oxaloacetate-derived amino acids and glucose than comparable plants that lack the variant PEPC enzyme. The disclosure also provides tools for production of plants that express the variant PEPC enzyme.

### CROSS-REFERENCE TO RELATED APPLICATIONS

This application is continuation of U.S. application Ser. No. 17/601,913, which is a national stage application under 35 U.S.C. § 371 of International Application No. PCT/US2020/27746, filed internationally on Apr. 10, 2020, which claims benefit of U.S. Provisional Application No. 62/832,727, filed Apr. 11, 2019, the disclosure of each of which is hereby incorporated by reference in its entirety.

### REFERENCE TO AN ELECTRONIC SEQUENCE LISTING

The content of the electronic sequence listing (677032002701SEQLIST.xml; Size: 217,791 bytes; and Date of Creation: Nov. 11, 2024) is herein incorporated by reference in its entirety.

### TECHNICAL FIELD

The present disclosure provides plants that express a variant phosphoenolpyruvate carboxylase (PEPC) enzyme. The plants have enhanced resistance to aluminum than comparable plants that lack the variant PEPC enzyme. In addition, the plants more effectively sequester carbon, extract phosphate, and produce oxaloacetate-derived amino acids and glucose than comparable plants that lack the variant PEPC enzyme. The disclosure also provides tools for production of plants that express the variant PEPC enzyme.

### BACKGROUND

Aluminum is considered to be a major limiting factor to crop growth in upwards of 50% of the world's arable land. A key approach for plants to adapt to aluminum toxic soils is to release aluminum chelating organic acids such as malate and citrate into the soil environment to chelate the aluminum to prevent it from being taken up into the root tissue. Prior work has found that increased release of malate and/or citrate into the rhizosphere increased the capability of plants to grow in aluminum toxic soils. This has been linked to increased capacity of plants to export these organic acids. However, attempts to engineer plants that have increased organic acid production have not been successful.

Accordingly, what is still needed in the art is another tool to increase aluminum resistance in plants. Also needed in the art are tools to increase organic acid release by plants into the soil so as to increase extraction of phosphate from the soil and to increase carbon sequestration in the soil. Further, tools for increasing production of oxaloacetate-derived amino acids and glucose by plants are desirable.

### SUMMARY

The present disclosure provides plants that express a variant phosphoenolpyruvate carboxylase (PEPC) enzyme. The plants have enhanced resistance to aluminum than comparable plants that lack the variant PEPC enzyme. In addition, the plants more effectively sequester carbon, extract phosphate, and produce oxaloacetate-derived amino acids and glucose than comparable plants that lack the variant PEPC enzyme. The disclosure also provides tools for production of plants that express the variant PEPC enzyme.

### BRIEF DESCRIPTION OF THE DRAWINGS

FIG. 1 shows the growth of Arabidopsis thaliana roots in hydroponic solution culture containing aluminum. Aluminum-dependent root growth inhibition was compared for wild type (wt) and mutant (alr-128) plants.

FIG. 2A shows aluminum-dependent callose accumulation in roots of wild type (wt) and mutant (alr-128) Arabidopsis thaliana plants. Roots of seedlings were exposed to a nutrient solution containing 75 μM AlCl₃ (pH 4.2) for 24 hours, except for the first panel in which no aluminum was added. The top row shows bright-field images, while the bottom row shows fluorescence images showing callose accumulation.

FIG. 2B shows patterns of aluminum accumulation by roots of wild type (wt) and mutant (alr-128) Arabidopsis thaliana plants. Seedlings grown in nutrient solution without aluminum were exposed to solution containing 25 μM AlCl₃ (pH 4.2) for 1 hour, except for the first panel in which no aluminum was added. Roots were stained with morin, which fluoresces when complexed with aluminum.

FIG. 3 shows pyruvate and malate exudation by roots of wild type (wt) and mutant (alr-108 and alr-128) Arabidopsis thaliana plants that were grown in a simple salt solution (pH 4.2) in the presence or absence of 2.7 μM AlCl₃.

FIG. 4 shows the relationship between pH and presence of aluminum ions.

FIG. 5A-F shows an alignment of amino acid sequences of various C3 phosphoenolpyruvate carboxylase (PEPC) enzymes. PEPC amino acid sequences are also set forth as: maize (SEQ ID NO:2), Arabidopsis (SEQ ID NO:1), soybean (SEQ ID NO:3), wheat (SEQ ID NO:4), barley (SEQ ID NO:5), rice (SEQ ID NO:6), sorghum (SEQ ID NO:7), and a consensus (SEQ ID NO:8). Variant PEPC enzymes of the present disclosure comprise an amino acid substitution in at least one position indicated by “\hat{ }” below the consensus sequence. A refined C3 PEPC consensus sequence is provided separately as SEQ ID NO:9.

FIG. 6 shows the enzymatic activity of wild type Arabidopsis thaliana PPC1 (SEQ ID NO:1) as compared to engineered PEPC enzymes. AtPPC1 is C3 PEPC. The graphs show increasing concentrations of the substrate phosphoenolpyruvate (x-axis) plotted against velocity (y-axis) at different concentrations of malate. The engineered Arabidopsis thaliana PPC1enzymes have a A651V substitution (alr-108), a G678S substitution (alr-128), a T778I substitution (alr-139), or a R886G substitution. Amino acid positions of engineered PEPC enzymes are relative to the amino acid sequences of both wild type Arabidopsis thaliana PPC1 (SEQ ID NO:1) and the consensus (SEQ ID NO:8).

FIG. 7 shows the enzymatic activity of wild type Zea mays PPC1 (SEQ ID NO:15) as compared to engineered PEPC enzymes. ZmPPC1 is a C4 PEPC. The graphs show increasing concentrations of the substrate phosphoenolpyruvate (x-axis) plotted against velocity (y-axis) at different concentrations of malate. The amino acid sequence of wild type Zea mays PPC1 has serine (S) at position 780 and glycine (G) at position 890 (SEQ ID NO:15), which corresponds to positions 776 and 886, respectively in the consensus (SEQ ID NO:8). The engineered Zea mays PPC1 enzymes have a A651V substitution (alr-108), a G678S substitution (alr-128), or a T778I substitution (alr-139), in addition to serine (S) at position 776 and glycine (G) at position 886. Amino acid positions of engineered PEPC enzymes are relative to the amino acid sequences of the consensus (SEQ ID NO:8).

### DEFINITIONS

To facilitate an understanding of the embodiments disclosed herein, a number of terms and phrases are defined below. Terms and abbreviations not defined should be accorded their ordinary meaning as used in the art.

As used herein and in the appended claims, the singular forms “a,” “an,” and “the” include plural references unless indicated otherwise. For example, “a” cell includes one or more cells. Likewise, “an” amino acid substitution refers to “at least one” amino acid substitution.

The term “about” as used herein in reference to a value, encompasses from 90% to 110% of that value (e.g., a pH of about 5 refers to a pH of 4.5 to 5.5 and includes a pH of 5.0).

Numeric ranges are inclusive of the numbers defining the range (e.g., a pH of from 2 to 5 encompasses a pH of 2, 3, 4 and 5).

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “isolated” means an object species (e.g., a nucleic acid) has been separated and/or recovered from components of its environment such that the object species is the predominant species present (i.e., on a molar basis it is more abundant than any other individual species in the composition). An “isolated” compound is at least 50% free, preferably at least 75% free, more preferably at least 90% free, and most preferably at least 95% free (e.g., 95%, 96%, 97%, 98%, or 99%) free from other compounds with which the compound of interest is typically associated.

As used herein, the term “phytotoxic substrate” refers to a growth substrate having a nanomolar or higher concentration of Al³⁺ ions and an acidic pH of from about 2 to about 5. In some embodiments, the phytotoxic substrate is soil.

As used herein, the term “aluminum resistance” refers to the ability of a plant to withstand contact with a phytotoxic substrate. Plants with aluminum resistance may be able to continuously grow and survive despite toxic levels of aluminum in the soil. In some embodiments, a plant with aluminum resistance may show minor symptoms caused by aluminum toxicity, such as root stunting and reduced water and nutrient uptake, but is still able to grow or produce fruit despite the aluminum toxicity.

The term “enhanced aluminum resistance” refers to an increased ability of a subject plant to tolerate contact with a phytotoxic substrate as compared to a control plant (e.g., another plant of the same genus and/or species) subject to the same conditions. In some embodiments, the increased aluminum resistance can be observed as an at least 10%, 15%, 20%, or 35% increase in root growth of a subject plant as compared to a control plant when both are grown in a salt solution comprising 25 μM AlCl₃.

As used herein, the terms “enhancing” and “increasing” relative to a parameter of interest (e.g., phosphate extraction, carbon sequestration, production of oxaloacetate-derived amino acids and glucose, etc.) refer to enlarging the magnitude of the parameter. One of skill in the art readily understands that this is generally as compared to conditions (e.g., control) that are otherwise the same except for a property of interest (e.g., expression of a variant PEPC enzyme). Depending upon the parameter measured, increasing may be from 2-fold to 2000-fold or over, or from any of 2, 5, 10, 20, 40 or 80-fold to any of 100, 200, 400, 800, 1600 or 3,200-fold over the control condition.

As used herein, the terms “phosphoenolpyruvate carboxylase” and “PEPC” refer to an enzyme found in plants and some bacteria. PEPC catalyzes the addition of bicarbonate to phosphoenolpyruvate (PEP) to form oxaloacetate and inorganic phosphate. PEPC is classified as EC 4.1.1.31 and CAS Registry Number: 9067-77-0.

The term “variant” when used in connection with PEPC refers to a PEPC with an amino acid sequence that differs from a wild type PEPC sequence of the same genus or species (e.g., not 100% identical). Preferably the variant PEPC is classifiable as EC 4.1.1.31 and CAS Registry Number: 9067-77-0. More preferably, the variant PEPC is less susceptible to feedback inhibition and/or has faster reaction kinetics.

In the context of two or more sequences (e.g., nucleic acid sequences or amino acid sequences) the terms “identical” and “identify” refer to the percentage of residues in a subject sequence that are identical to residues in a reference sequence, after aligning the sequences and introducing gaps, if necessary, to achieve the maximum percent sequence identity. Conservative substitutions are not considered as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

### DETAILED DESCRIPTION

### I. Introduction

A previous mutagenesis approach using Arabidopsis as a model system resulted in several mutant plants that could grow robustly in an aluminum toxic environment (Larsen et al., Plant Physiol, 117: 9-18, 1998). Although the phenotype of the mutants was assessed, the genotype of the mutants was not heretofore determined.

A key approach for plants to adapt to aluminum toxic soils and/or acidic soils (e.g., soils with a pH of 5 or lower; a pH of 5, 4.5, 4, 3.5, 3, 2.5, or 2) is to release aluminum-chelating organic acids, such as malate and citrate, into the soil environment to chelate the aluminum and prevent it from being taken up into the root tissue. An effort was made to identify the mutations that are responsible for the aluminum-resistance phenotype of Arabidopsis thaliana mutants alr-108, alr-128 and alr-139. A whole genome sequencing project was undertaken for alr-128 in which a genomic library from the mutant was generated and analyzed. This approach revealed a homozygous mutation in Atlg53310, in an amino acid that is strictly conserved amongst all phosphoenolpyruvate carboxylases (PEPCs) identified, but has no known role in PEPC function. Following this, Atlg53310 was sequenced for both alr-108 and alr-139, with each of these also having mutations that lead to amino acid substitutions in invariant or highly conserved positions in PEPCs in general.

Phosphoenolpyruvate carboxylase (PEPC) is an enzyme that is key to production of oxaloacetate as a means to replenish the tricarboxylic acid (TCA) cycle in plants. PEPC has a similar role to pyruvate carboxylase in animals, both of which are responsible for generating oxaloacetate for replenishing TCA cycle intermediates that are removed for processes such as amino acid production or fatty acid biosynthesis. Work has been performed to try to link PEPC overexpression to increases in aluminum resistance, but wild-type PEPC overexpression alone has resulted in only marginal increases in aluminum resistance.

Two isoforms of PEPC are C3 PEPC and C4 PEPC. The C3 PEPC is the key enzyme in the classical C3 non-photosynthetic pathway, which is the main form of PEPC in plants. The C3 PEPC has a malate binding site that serves to allosterically control C3 PEPC activity by malate feedback inhibition. In general, the PEPC present in the roots of plants is the C3 PEPC. In contrast, the C4 PEPC, which is strictly linked to C4 photosynthesis in shoots of a limited number of plant species, has reduced malate-dependent feedback and thus, is less affected allosterically by malate. In SEQ ID NO:2 (maize C3 PEPC): i) A at position 770 is a hallmark for C3 and would be S if C4; and ii) R at position 880 is a hallmark for C3 and would be G if C4. In SEQ ID NO:15 (maize C4 PEPC): i) S at position 780 is a hallmark for C4 and would be A if C3; and ii) G at position 890 is a hallmark for C4 and would be R if C3. Positions 770 and 880 of maize C3 PEPC and positions 780 and 890 of maize C4 PEPC correspond to positions 776 and 886 respectively in the consensus sequence of SEQ ID NO:8.

The present disclosure provides compositions and methods that modify the function and behavior of PEPC in roots to enhance aluminum resistance in plants. As described further herein, increasing PEPC activity in roots confers increased malate production in plants and consequently, provides aluminum resistance. The present disclosure further provides compositions and methods that modify the function and behavior of PEPC in other plant parts to enhance photosynthesis in plants. As described further herein, increasing PEPC activity in above-ground plant parts confers increased glucose production in plants.

Additionally, a number of genes have been found to be differentially expressed in citrus plants grown in the presence of a high level of aluminum and a low level of phosphorus (Yang et al., Mol Biol Rep, 39: 6353-6366, 2012). More recently, phosphoenolpyruvate carboxylase (PEPC) expression was found to be induced in soybeans subjected to various abiotic stresses (Wang et al., Scientific Reports, 6: 38448, 2016). However, prior to development of the present disclosure, variant root PEPC enzymes conferring aluminum-resistance had not been identified.

### II. Variant Phosphoenolpyruvate Carboxylase (PEPC)

Increasing the production of aluminum-chelating organic acids, such as malate, in a plant may enhance the plant's aluminum resistance. Since phosphoenolpyruvate carboxylase (PEPC) in plants catalyzes the addition of bicarbonate to phosphoenolpyruvate (PEP) to form oxaloacetate, which is a precursor of malate, improved PEPC activity is likely to increase oxaloacetate production resulting in increased malate levels. Improving the activity of PEPC, which is present in the roots of plants, may be particularly beneficial to enhancing aluminum resistance in plants, especially in plants grown in soil with a high aluminum concentration and/or an acidic pH (e.g., soils with a micromolar or higher levels of Al³⁺ and/or pH from 2-5).

Accordingly, in one aspect, the present disclosure provides a variant phosphoenolpyruvate carboxylase (PEPC) having improved activity such that its expression in a plant leads to increased production of oxaloacetate and malate, which in turn results in enhanced aluminum resistance in the plant. The improved activity of the variant PEPC may be achieved by reducing the enzyme's sensitivity to allosteric feedback inhibition by malate and/or by increasing the enzyme's active site activity.

Accordingly, the variant PEPC of the present disclosure may contain one or more amino acid substitutions that are conducive to improved PEPC activity. Some preferred and alternative substitutions are listed in Table I. Throughout the present disclosure and unless indicated to the contrary, amino acid positions are numbered relative to SEQ ID NO:8 as determined when the amino acid sequence of a PEPC enzyme of interest is aligned to SEQ ID NO:8 using a pairwise alignment algorithm. For instance, the amino acid sequence of wild type Zea mays PPC1 has serine (S) at position 780 and glycine (G) at position 890 (SEQ ID NO:15), which corresponds to positions 776 and 886, respectively in the consensus sequence (SEQ ID NO:8). The numbering of the refined consensus sequence of SEQ ID NO:9 is equivalent to the consensus sequence of SEQ ID NO:8. Thus, amino acid positions numbered relative to SEQ ID NO: 8 are also numbered relative to SEQ ID NO:9.

TABLE I
Favored Substitutions

| Original Residue | Preferred Substitutions | Other Substitutions |
|---|---|---|
| Arg637 (R637) | Gly (G) or Ala (A) or Val (V) | Ile (I) or Leu (L) or |
| | | Met (M) |
| Ala651 (A651) | Val (V) or Ile (I) or Leu (L) | Met (M) or Ser (S) |
| | | or Thr (T) |
| Gln675 (Q675) | Gly (G) or Ala (A) or Val (V) | Ile (I) or Leu (L) or |
| | | Met (M) |
| Gly678 (G678) | Ser (S) or Thr (T) | Val (V) or Ile (I) or |
| | | Leu (L) |
| Ala776 (A776) | Ser (S) or Thr (T) | Val (V) or Ile (I) or |
| | | Leu (L) |
| Thr778 (T778) | Ile (I) or Leu (L) or Met (M) | Gly (G) or Ala (A) |
| | | or Val (V) |
| Lys831 (K831) | Gly (G) or Ala (A) or Val (V) | Ile (I) or Leu (L) or |
| | | Met (M) |
| Arg886 (R886) | Gly (G) or Ala (A) or Val (V) | Ile (I) or Leu (L) or |
| | | Met (M) |
| Arg890 (R890) | Gly (G) or Ala (A) or Val (V) | Ile (I) or Leu (L) or |
| | | Met (M) |
| Asn965 (N965) | Gly (G) or Ala (A) or Val (V) | Ile (I) or Leu (L) or |
| | | Met (M) |

C3 PEPC activity is strictly controlled by the allosteric regulator malate, which when accumulated to high levels, results in strong inhibition of PEPC activity in roots. There are several amino acids that are directly involved in malate binding at the allosteric pocket of PEPC. In some embodiments, the present disclosure provides compositions and methods for increasing PEPC activity by reducing the enzyme's sensitivity to feedback inhibition by malate. Arabidopsis thaliana mutants alr-108 and alr-128, which contain amino acid substitutions A651V and G678S, respectively, relative to the sequence of SEQ ID NO:1, are both thought to alter how the malate binding site communicates with the active site of PEPC. The malate binding site of PEPC also includes several positively charged amino acids (e.g., Arg and Lys) that function to bind the negatively charged malate. These positively charge amino acids may be changed to alter the association of malate in the malate binding site and consequently, relieve the feedback inhibition of PEPC activity by malate.

In some embodiments, one or more positively charged amino acids in the malate binding site of PEPC may be substituted with an uncharged or negatively charged amino acid (e.g., Ala, Gly, Val, Leu, Ile, Met, Asp, or Glu), to reduce malate binding and consequently to reduce the sensitivity of the PEPC to feedback inhibition by malate. In some embodiments, amino acids in the malate binding site of PEPC that may be mutated to reduce the sensitivity of PEPC to feedback inhibition by malate include, but are not limited to, R637, A651, Q675, G678, K831, R886, R890, and N965, relative to the sequence of SEQ ID NO:1. In some embodiments, uncharged or negatively charged amino acids (e.g., Ala, Gly, Val, Leu, Ile, Met, Asp, or Glu) may be present in one or more positions selected from 637, 651, 675, 678, 831, 886, 890 or 965. In certain embodiments, amino acid substitutions in PEPC that may reduce the sensitivity of PEPC to feedback inhibition by malate include, but are not limited to, A651V, G678S, and R886G.

PEPC may also be engineered to increase oxaloacetate production by increasing the enzyme's active site activity. The active site of the PEPC may be modified to improve the kinetics of the enzyme (e.g., increasing the binding affinity of the enzyme to its substrate phosphoenolpyruvate, and/or increasing other aspects of the catalytic efficacy of the enzyme such as its reaction rate). As described in Example 1, A. thaliana mutant alr-139 contains amino acid substitution T778I, relative to the sequence of SEQ ID NO:1, the position of which maps to the active site of PEPC.

In some embodiments, one or more amino acids in the active site of PEPC may be altered to increase PEPC activity. In some embodiments, one or more polar amino acids (e.g., Thr, Ser, Cys, Asn, and Gln) in the active site of PEPC may be substituted with a nonpolar amino acid (e.g., Gly, Ala, Val, Leu, Met, and Ile). In certain embodiments, amino acid substitutions that may increase enzymatic activity of PEPC include but are not limited to T778I and/or A776S, relative to the sequence of SEQ ID NO:1.

In some embodiments, the variant phosphoenolpyruvate carboxylase (PEPC) comprises at least one amino acid substitution at a position corresponding to one or more of residues A651, G678, A776, T778, and R886, in the consensus sequence of SEQ ID NO:8, where the amino acid sequence of the variant is at least 90%, 91%, 92%, 93%, 94% 95%, 96%, 97%, 98%, or 99% identical to the consensus sequence of SEQ ID NO:8, and where the amino acid sequence of the variant does not consist of SEQ ID NO:10, SEQ ID NO:11, or SEQ ID NO:12. In some embodiments, the variant PEPC comprises at least one further amino acid substitution at a position corresponding to one or more of residues R637, X675, K831, R890 and N965 in the consensus sequence of SEQ ID NO:8, where X675 is Q675 or H675. In some embodiments, the variant PEPC comprises at least one amino acid substitution at a position corresponding to one or more of residues A651, G678, and T778 in the consensus sequence of SEQ ID NO:8. In some embodiments, the variant PEPC further comprises an amino acid substitution at a position corresponding to one or both of A776 and R886 in the consensus sequence of SEQ ID NO:8. In some embodiments, the variant PEPC comprises one or more amino acid substitutions selected from the group consisting of A651V, G678S, A776S, T778I, and R886G. In some embodiments, the variant PEPC comprises one or more amino acid substitutions selected from the group consisting of A651V, G678S, and T778I. In some embodiments, the variant PEPC further comprises an amino acid substitution selected from the group consisting of one or both of A776S and R886G. In some embodiments, the amino acid sequence of the variant is at least 99% identical to SEQ ID NO:9.

Two PEPC sequences are substantially identical if their amino acid sequences have at least 50% identity (e.g., at least 50%, 55%, 60%, 65%, 70%, 75%, 80%, 85%, 90%, 95%, 99% or 100% identity over a specified region, or, when not specified, over their entire sequences), when compared and aligned for maximum correspondence over a comparison window or designated region. As pertains to the present disclosure and claims, the BLASTP sequence comparison algorithm using default parameters is used align amino acid sequences for determination of sequence identity.

Algorithms that are suitable for determining percent sequence identity and sequence similarity are the BLAST and BLAST 2.0 algorithms, described in Altschul et al., J Mol Biol, 215: 403-410, 1990; and Altschul et al., Nucleic Acids Res. 25: 3389-3402, 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89: 10915 (1989)).

### III. Nucleic Acids and Expression Cassettes

### Nucleic Acids

In some embodiments, the present disclosure is related to a nucleic acid encoding a variant phosphoenolpyruvate carboxylase (PEPC) of any one of the preceding embodiments. In some embodiments, the present disclosure is related to an isolated nucleic acid encoding a variant phosphoenolpyruvate carboxylase (PEPC) comprising at least one amino acid substitution at a position corresponding to one or more of residues A651, G678, A776, T778, and R886, in the consensus sequence of SEQ ID NO:8, where the amino acid sequence of the variant is at least 95% identical to the consensus sequence of SEQ ID NO:8, and where the amino acid sequence of the variant does not consist of SEQ ID NO:10, SEQ ID NO:11, or SEQ ID NO:12. The nucleic acid encoding a variant PEPC of the present disclosure may be of any nucleic acid type, including RNA, such as messenger RNA (mRNA), and DNA, such as complementary DNA (cDNA), genomic DNA (gDNA), and synthetic DNA.

In another aspect, the present disclosure provides an expression cassette comprising a promoter operably linked to a nucleic acid encoding a variant PEPC of any of the preceding embodiments. As used herein, an “expression cassette” refers to a nucleic acid construct that, when introduced into a host cell, results in transcription and/or translation of an RNA or polypeptide, respectively.

In some embodiments, the expression cassette of the present disclosure comprises a promoter operably linked to the nucleic acid encoding the variant PEPC. The promoter may be heterologous to the nucleic acid. In some embodiments, the promoter may be inducible. In some embodiments, the promoter may plant tissue-specific (e.g., phloem-specific, tuber-specific, root-specific, stem-specific, trunk-specific, or leaf-specific).

Any promoters well known in the art may be used to drive the expression of a variant PEPC in plants. Any organ may be targeted, such as shoot vegetative organs/structures (e.g. leaves, stems, and tubers), roots, flowers and floral organs/structures (e.g. bracts, sepals, petals, stamens, carpels, anthers and ovules), seed (including embryo, endosperm, and seed coat) and fruit. Alternatively, the nucleic acid encoding a variant PEPC described herein may be expressed specifically in certain cell and/or tissue types within one or more organs (e.g., guard cells in leaves using a guard cell-specific promoter). Alternatively, the nucleic acid encoding a variant PEPC described herein may be expressed constitutively (e.g., using the CaMV 35S promoter).

To use a nucleic acid encoding a variant PEPC described herein in the above techniques, recombinant DNA vectors suitable for transformation of plant cells may be prepared. Techniques for transforming a wide variety of higher plant species are well described in the technical and scientific literature (see, e.g., Weising et al., Ann. Rev. Genet. 22: 421-477, 1988). A DNA sequence coding for the variant PEPC preferably may be combined with transcriptional and translational initiation regulatory sequences that direct the transcription of the sequence from the gene in the intended tissues of the transformed plant.

For example, a plant promoter fragment may be employed to direct expression of the variant PEPC in all tissues of a transgenic plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the variant PEPC in a specific tissue (tissue-specific promoters) or may be otherwise under more precise environmental control (inducible promoters). Examples of tissue-specific promoters under developmental control include promoters that initiate transcription only in certain tissues, such as roots, phloem, tubers, stems, trunks, leaves, or guard cells. In particular embodiments, a plant promoter may be employed to direct expression of the variant PEPC in root tissues of a plant. Examples of environmental conditions that may affect transcription by inducible promoters include, but are not limited to, anaerobic conditions, elevated temperature, elevated toxic metal concentration in soil such as aluminum, and presence of light.

In some embodiments, the promoter is heterologous to the nucleic acid encoding the variant PEPC of the present disclosure. As used herein, a “heterologous” promoter refers to a promoter is from a different origin than the nucleic acid encoding the variant PEPC. Thus, a promoter that has been isolated from an organism different from that of the nucleic acid encoding the variant PEPC is considered heterologous with respect to the nucleic acid encoding the variant PEPC; a promoter that has been isolated from a gene that is different from that of the nucleic acid encoding the variant PEPC is also considered heterologous with respect to the nucleic acid encoding the variant PEPC.

### Constitutive Promoters

In some embodiments, the expression cassette of the present disclosure comprises a constitutive promoter directing expression of the nucleic acid encoding the variant PEPC in all transformed cells or tissues, e.g., as those of a transgenic plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313: 810-812 (1985)). The CaMV 35S promoter can be particularly useful due to its activity in numerous diverse plant species (Benfey and Chua, Science 250: 959-966 (1990); Futterer et al., Physiol. Plant 79: 154 (1990); Odell et al., supra, 1985). A tandem 35S promoter, in which the intrinsic promoter element has been duplicated, confers higher expression levels in comparison to the unmodified 35S promoter (Kay et al., Science 236: 1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mol. Biol. 14: 433 (1990); An, Plant Physiol. 81: 86 (1986)).

Additional constitutive regulatory elements including those for efficient expression in monocots also are known in the art, for example, the pEmu promoter and promoters based on the rice Actin-1 5′ region (Last et al., Theor. Appl. Genet. 81: 581 (1991); Mcelroy et al., Mol. Gen. Genet. 231: 150 (1991); Mcelroy et al., Plant Cell 2: 163 (1990)). Chimeric regulatory elements, which combine elements from different genes, also can be useful for ectopically expressing a nucleic acid molecule encoding a variant PEPC described herein (Comai et al., Plant Mol. Biol. 15: 373 (1990)).

Other examples of constitutive promoters include the 1′- or 2′-promoter derived from T-DNA of Agrobacterium tumafaciens (see, e.g., Mengiste (1997) supra; O'Grady (1995) Plant Mol. Biol. 29: 99-108); actin promoters, such as the Arabidopsis actin gene promoter (see, e.g., Huang (1997) Plant Mol. Biol. 1997 33: 125-139); alcohol dehydrogenase (Adh) gene promoters (see, e.g., Millar (1996) Plant Mol. Biol. 31: 897-904); ACT11 from Arabidopsis (Huang et al. Plant Mol. Biol. 33: 125-139 (1996)), Cat3 from Arabidopsis (GenBank No. U43147, Zhong et al., Mol. Gen. Genet. 251: 196-203 (1996)), the gene encoding stearoyl-acyl carrier protein desaturase from Brassica napus (Genbank No. X74782, Solocombe et al. Plant Physiol. 104: 1167-1176 (1994)), GPc1 from maize (GenBank No. X15596, Martinez et al. J. Mol. Biol 208: 551-565 (1989)), Gpc2 from maize (GenBank No. U45855, Manjunath et al., Plant Mol. Biol. 33: 97-112 (1997)), other transcription initiation regions from various plant genes known to those of skill. See also Holtorf Plant Mol. Biol. 29: 637-646 (1995).

### Inducible Promoters

In some embodiments, the expression cassette of the present disclosure comprises an inducible promoter directing expression of the nucleic acid encoding the variant PEPC under the influence of changing environmental conditions or developmental conditions. Examples of environmental conditions that may affect transcription by inducible promoters include anaerobic conditions, elevated temperature, drought, toxic metals and/or the presence of light. Such promoters are referred to herein as “inducible” promoters. In some embodiments, an inducible promoter is one that is induced by one or more environmental stressors, including but not limited to, drought, freezing cold, toxic metals, and high salt. For example, the disclosure can incorporate a drought-specific promoter such as a drought-inducible promoter of maize (e.g., the maize rab 17 drought-inducible promoter (Vilardell et al. (1991) Plant Mol. Biol. 17: 985-993; Vilardell et al. (1994) Plant Mol. Biol. 24: 561-569)); or alternatively a cold, drought, and high salt inducible promoter from potato (Kirch (1997) Plant Mol. Biol. 33: 897-909) or from Arabidopsis (e.g., the rd29A promoter (Kasuga et al. (1999) Nature Biotechnology 17: 287-291). Other environmental stress-inducible promoters include promoters from the following genes: Rab21, Wsi18, Lea3, Uge1, Dip1, and R1G1B in rice (Yi et al. (2010) Planta 232: 743-754).

In some embodiments, the inducible promoter is a stress-inducible promoter (e.g., a drought-, cold-, or salt-inducible promoter) that comprises a dehydration-responsive element (DRE) and/or an ABA-responsive clement (ABRE), including but not limited to the rd29A promoter.

Alternatively, plant promoters that are inducible upon exposure to plant hormones, such as auxins, are used to express the nucleic acid encoding the variant PEPC. For example, the disclosure can use the auxin-response elements E1 promoter fragment (AuxREs) in the soybean (Glycine max L.) (Liu (1997) Plant Physiol. 115: 397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37: 906-913); a plant biotin response element (Streit (1997) Mol. Plant Microbe Interact. 10: 933-937); and, the promoter responsive to the stress hormone abscisic acid (Sheen (1996) Science 274: 1900-1902).

Plant promoters inducible upon exposure to chemical reagents that may be applied to the plant, such as herbicides or antibiotics, are also useful for expressing the nucleic acid encoding the variant PEPC. For example, the maize In2-2 promoter, activated by benzenesulfonamide herbicide safeners, can be used (De Veylder (1997) Plant Cell Physiol. 38: 568-577); application of different herbicide safeners induces distinct gene expression patterns, including expression in the root, hydathodes, and the shoot apical meristem. A variant PEPC coding sequence can also be under the control of, e.g., a tetracycline-inducible promoter, e.g., as described with transgenic tobacco plants containing the Avena sativa L. (oat) arginine decarboxylase gene (Masgrau (1997) Plant J. 11: 465-473); or, a salicylic acid-responsive element (Stange (1997) Plant J. 11: 1315-1324; Uknes et al., Plant Cell 5: 159-169 (1993); Bi et al., Plant J. 8: 235-245 (1995)).

Examples of useful inducible regulatory elements include copper-inducible regulatory elements (Mett et al., Proc. Natl. Acad. Sci. USA 90: 4567-4571 (1993); Furst et al., Cell 55: 705-717 (1988)); tetracycline and chlor-tetracycline-inducible regulatory elements (Gatz et al., Plant J. 2: 397-404 (1992); Röder et al., Mol. Gen. Genet. 243: 32-38 (1994); Gatz, Meth. Cell Biol. 50: 411-424 (1995)); ecdysone inducible regulatory elements (Christopherson et al., Proc. Natl. Acad. Sci. USA 89: 6314-6318 (1992); Kreutzweiser et al., Ecotoxicol. Environ. Safety 28: 14-24 (1994)); heat shock inducible regulatory elements (Takahashi et al., Plant Physiol. 99: 383-390 (1992); Yabe et al., Plant Cell Physiol. 35: 1207-1219 (1994); Ueda et al., Mol. Gen. Genet. 250: 533-539 (1996)); and lac operon elements, which are used in combination with a constitutively expressed lac repressor to confer, for example, IPTG-inducible expression (Wilde et al., EMBO J. 11: 1251-1259 (1992)). An inducible regulatory element useful in the transgenic plants of the present disclosure also can be, for example, a nitrate-inducible promoter derived from the spinach nitrite reductase gene (Back et al., Plant Mol. Biol. 17: 9 (1991)) or a light-inducible promoter, such as that associated with the small subunit of RuBP carboxylase or the LHCP gene families (Feinbaum et al., Mol. Gen. Genet. 226: 449 (1991); Lam and Chua, Science 248: 471 (1990)).

### Tissue-Specific Promoters

In some embodiments, the expression cassette of the present disclosure comprises a tissue-specific promoter directing expression of the nucleic acid encoding the variant PEPC in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue, or epidermis or mesophyll. Reproductive tissue-specific promoters may be, e.g., ovule-specific, embryo-specific, endosperm-specific, integument-specific, seed and seed coat-specific, pollen-specific, petal-specific, sepal-specific, or some combination thereof. In some embodiments, the promoter is cell-type specific (e.g., guard cell-specific, bundle sheath cell-specific, etc.). In particular embodiments, the promoter may direct expression of the nucleic acid encoding the variant PEPC in a root tissue of the plant.

Epidermal-specific promoters include, for example, the Arabidopsis LTP1 promoter (Thoma et al. (1994) Plant Physiol. 105(1): 35-45), the CER1 promoter (Aarts et al. (1995) Plant Cell 7: 2115-27), and the CER6 promoter (Hooker et al. (2002) Plant Physiol 129: 1568-80), and the orthologous tomato LeCER6 (Vogg et al. (2004) J. Exp Bot. 55: 1401-10).

Guard cell-specific promoters include, for example, the DGP1 promoter (Li et al. (2005) Science China C Life Sci. 48: 181-186).

Other tissue-specific promoters include seed promoters. Suitable seed-specific promoters are derived from the following genes: MAC1 from maize (Sheridan (1996) Genetics 142: 1009-1020); Cat3 from maize (GenBank No. L05934, Abler (1993) Plant Mol. Biol. 22: 10131-1038); vivparous-1 from Arabidopsis (Genbank No. U93215); atmycl from Arabidopsis (Urao (1996) Plant Mol. Biol. 32: 571-57; Conceicao (1994) Plant 5: 493-505); napA from Brassica napus (GenBank No. J02798, Josefsson (1987) JBL 26: 12196-1301); and the napin gene family from Brassica napus (Sjodahl (1995) Planta 197: 264-271).

A variety of promoters specifically active in vegetative tissues, such as leaves, stems, roots and tubers, can also be used to express nucleic acid encoding a variant PEPC described herein. For example, promoters controlling patatin, the major storage protein of the potato tuber, can be used, sec, e.g., Kim (1994) Plant Mol. Biol. 26: 603-615; Martin (1997) Plant J. 11: 53-62. The ORF13 promoter from Agrobacterium rhizogenes that exhibits high activity in roots can also be used (Hansen (1997) Mol. Gen. Genet. 254: 337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28: 137-144); the curculin promoter active during taro corm development (de Castro (1992) Plant Cell 4: 1549-1559) and the promoter for the tobacco root-specific gene TobRB7, whose expression is localized to root meristem and immature central cylinder regions (Yamamoto (1991) Plant Cell 3: 371-382).

Leaf-specific promoters, such as the ribulose biphosphate carboxylase (RBCS) promoters, can also be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415: 91-95). A ribulose bisphosphate carboxylase promoters expressed almost exclusively in mesophyll cells in leaf blades and leaf sheaths at high levels, described by Matsuoka (1994) Plant J. 6: 311-319, can be used. Another leaf-specific promoter is the light harvesting chlorophyll a/b binding protein gene promoter, see, e.g., Shiina (1997) Plant Physiol. 115: 477-483; Casal (1998) Plant Physiol. 116: 1533-1538. The Arabidopsis thaliana myb-related gene promoter (Atmyb5) described by Li (1996) FEBS Lett. 379: 117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11: 1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86: 423-433; and, Long (1996) Nature 379: 66-69; can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7: 517-527). Also useful are kn1-related genes from maize and other species which show meristem-specific expression, see, e.g., Granger (1996) Plant Mol. Biol. 31: 373-378; Kerstetter (1994) Plant Cell 6: 1877-1887; Hake (1995) Philos. Trans. R. Soc. Lond. B. Biol. Sci. 350: 45-51. For example, the Arabidopsis thaliana KNAT1promoter (see, e.g., Lincoln (1994) Plant Cell 6: 1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, the nucleic acid encoding the variant PEPC is expressed through a transposable element. This allows for constitutive, yet periodic and infrequent expression of the constitutively active polypeptide. The disclosure also provides for use of tissue-specific promoters derived from viruses including, e.g., the tobamovirus subgenomic promoter (Kumagai (1995) Proc. Natl. Acad. Sci. USA 92: 1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31: 1129-1139).

### Expression Vectors

In some embodiments, the present disclosure provides for expression vectors comprising an expression cassette of any one of the preceding embodiments. As used herein, an “expression vector” refers to a vector comprising a recombinant nucleic acid comprising expression control sequences operatively linked to a nucleic acid to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In some embodiments, the expression vector is a plasmid.

### Host Cells

In some embodiments, the present disclosure provides host cells comprising an expression cassette of any one of the preceding embodiments. The host cell may be of any type of cell. In some embodiments, the host cell is prokaryotic or eukaryotic. In some embodiments, the host cell is a bacterial cell, a yeast cell, a mammalian cell, or a plant cell. In some particular embodiments, the host cell is a plant cell.

### Transgenic Plants

In other aspects, transgenic plants containing a host cell of the present disclosure are provided. As used herein, a “transgenic plant” refers to a plant that has incorporated a heterologous or exogenous nucleic acid, i.e., a nucleotide sequence that is not present in the native (non-transgenic or “untransformed”) plant or plant cell. “Transgenic” is used herein to include any cell, cell line, callus, tissue, plant part or plant, the genotype of which has been altered by the presence of heterologous nucleotide sequence including those transgenics initially so altered as well as those created by sexual crosses or asexual propagation from the initial transgenic plant. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. In some embodiments, a transgenic plant is generated that contains a complete or partial sequence of a nucleic acid that is derived from a species other than the species of the transgenic plant. It should be recognized that transgenic plants encompass the plant or plant cell in which the expression cassette is introduced as well as progeny of such plants or plant cells that contain the expression cassette, including the progeny that have the expression cassette stably integrated in a chromosome. In some embodiments, the transgenic plant expresses the variant PEPC. In some embodiments, the transgenic plant has enhanced aluminum resistance as compared to a control plant of the same species that does not express the variant PEPC.

### IV. Methods of Producing Plants

In other aspects, the present disclosure relates generally to methods of producing a plant having enhanced aluminum resistance by expressing a variant phosphoenolpyruvate carboxylase (PEPC) in the plant. In some embodiments, the expression of a variant PEPC in the plant is achieved by means of plant transformation. In some embodiments, the expression of a variant PEPC in the plant is achieved by means of genome editing, such as the CRISPR/Cas method.

### Plant Transformation

In one aspect, the expression of a variant PEPC of the present disclosure in the plant is achieved by means of plant transformation. For example, in some embodiments, the present disclosure provides a method for producing a plant expressing a variant phosphoenolpyruvate carboxylase (PEPC), comprising: (a) introducing an expression cassette of any of the preceding embodiments into a plant cell to form a transformed plant cell; and (b) regenerating a plant from the transformed plant cell, where the plant expresses the variant PEPC and has enhanced aluminum resistance as compared to a control plant of the same species that does not express the variant PEPC.

As used herein, the term “plant transformation” encompasses all techniques by which a heterologous nucleic acid may be introduced into a plant cell. As used herein, a “heterologous nucleic acid” refers to a nucleic acid or a portion thereof that is not native to the host cell in nature, such as an artificially assembled expression cassette. A host cell or organism containing the heterologous nucleic acid stably integrated into the genome is referred to as a “transformed” cell or organism.

An expression cassette of the present disclosure may be introduced into the genome of the desired plant host by a variety of conventional techniques. For example, the expression cassette may be introduced directly into the genomic DNA of the plant cell using techniques such as electroporation and microinjection of plant cell protoplasts, or the expression cassette can be introduced directly to plant tissue using ballistic methods, such as DNA particle bombardment. Alternatively, the expression cassette may be combined with suitable T-DNA flanking regions and introduced into a conventional Agrobacterium host vector. The virulence functions of the Agrobacterium host will direct the insertion of the construct and adjacent marker into the plant cell DNA when the cell is infected by the bacteria. While transient expression of the constitutively active PEPC is encompassed by the disclosure, generally, expression of a construct of the present disclosure will be from insertion of expression cassettes into the plant genome, e.g., such that at least some plant offspring also contain the integrated expression cassette. Microinjection techniques are also useful for this purpose. These techniques are well known in the art and thoroughly described in the literature. The introduction of expression cassettes using polyethylene glycol precipitation is described in Paszkowski et al. EMBO J. 3: 2717-2722 (1984). Electroporation techniques are described in Fromm et al. Proc. Natl. Acad. Sci. USA 82: 5824 (1985). Ballistic transformation techniques are described in Klein et al. Nature 327: 70-73 (1987). Agrobacterium-mediated transformation techniques, including disarming and use of binary vectors, are well described in the scientific literature. See, for example, Horsch et al. Science 233: 496-498 (1984), and Fraley et al. Proc. Natl. Acad. Sci. USA 80: 4803 (1983).

The following are representative publications disclosing plant transformation protocols that can be used to genetically transform the following plant species: maize (U.S. Pat. Nos. 5,177,010 and 5,981,840); soybean (U.S. Pat. Nos. 5,416,011; 5,569,834; 5,824,877; 5,563,04455 and 5,968,830); wheat (Ortiz et al., 1996, Plant Cell Rep. 15, 877); barley (U.S. Pat. No. 6,100,447); rice (Alam et al., 1999, Plant Cell Rep. 18, 572); sorghum (Guo et al., 2015, Methods Mol Biol 1223, 181-188; Howe et al., Plant Cell Rep 25(8): 784-791, 2006). Transformation of other species is also contemplated by the disclosure. Suitable methods and protocols for transformation of other species are available in the scientific literature and known to those of skill in the art.

Transformed plant cells derived by any of the above transformation techniques may be cultured to regenerate a whole plant that possesses the transformed genotype and thus the desired phenotype, e.g., aluminum resistance. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof (see, e.g., Klee et al., Ann. Rev. of Plant Phys. 38: 467-486, 1987).

One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed. The expression cassettes and other constructs of the present disclosure can be used to confer aluminum resistance on essentially any plant. In some embodiment, the plant is a grain-, vegetable-, or fruit-producing plant.

Those of skill will recognize that a number of plant species can be used as models to predict the phenotypic effects of transgene expression in other plants. For example, it is well recognized that Arabidopsis plants are useful models of transgene expression. In some embodiments, the plants of the present disclosure have enhanced PEPC-mediated phenotypes, for example enhanced aluminum resistance, as compared to a control plant of the same species that does not express the variant PEPC.

### CRISPR/Cas

In another aspect, the expression of a variant PEPC of the present disclosure in the plant is achieved by means of genome editing, such as the CRISPR/Cas method.

Plant gene manipulations can now be precisely tailored in non-transgenic organisms using the CRISPR/Cas9 genome editing method. In this bacterial antiviral and transcriptional regulatory system, a complex of two small RNAs—the CRISPR-RNA (crRNA) and the trans-activating crRNA (tracrRNA)—directs the nuclease (Cas9) to a specific DNA sequence complementary to the crRNA (Jinek, M., et al. Science 337, 816-821 (2012)). Binding of these RNAs to Cas9 involves specific sequences and secondary structures in the RNA. The two RNA components can be simplified into a single element, the single guide-RNA (sgRNA), which is transcribed from a cassette containing a target sequence defined by the user (Jinek, M., et al. Science 337, 816-821 (2012)). This system has been used for genome editing in humans, zebrafish, Drosophila, mice, nematodes, bacteria, yeast, and plants (Hsu, P. D., et al., Cell 157, 1262-1278 (2014)). In this system the nuclease creates double stranded breaks at the target region programmed by the sgRNA. These can be repaired by non-homologous recombination, which often yields inactivating mutations. The breaks can also be repaired by homologous recombination, which enables the system to be used for gene targeted gene replacement (Li, J. -F., et al. Nat. Biotechnol. 31, 688-691, 2013; Shan, Q., et al. Nat. Biotechnol. 31, 686-688, 2013). In some embodiments of the methods in the present disclosure, a gene encoding a wild-type or endogenous PEPC in a plant may be modified using the CAS9/CRISPR system to match the nucleic acid sequence encoding a variant PEPC described herein.

Thus, in some embodiments, instead of generating a transgenic plant, a wild-type PEPC coding sequence in a plant or plant cell can be altered in situ to generate a plant or plant cell carrying a nucleic acid encoding a variant PEPC described herein of the present disclosure. The CRISPR/Cas system has been modified for use in prokaryotic and eukaryotic systems for genome editing and transcriptional regulation. The “CRISPR/Cas” system refers to a widespread class of bacterial systems for defense against foreign nucleic acid. CRISPR/Cas systems are found in a wide range of cubacterial and archacal organisms. CRISPR/Cas systems include type I, II, and III sub-types. Wild-type type II CRISPR/Cas systems utilize the RNA-mediated nuclease, Cas9 in complex with guide and activating RNA to recognize and cleave foreign nucleic acid. Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci U S A. 2013 Sep. 24;110 (39): 15644-9; Sampson et al., Nature. 2013 May 9; 497(7448): 254-7; and Jinck, et al., Science. 2012 Aug. 17; 337(6096): 816-21.

Accordingly, in some embodiments, the present disclosure provides a method for producing a plant expressing a variant phosphoenolpyruvate carboxylase (PEPC), comprising: (a) introducing a clustered regularly interspaced short palindromic repeats (CRISPR) associated protein (Cas9) genome-editing system into a plant cell to form a transformed plant cell comprising a nucleic acid of any one of the preceding embodiments; and (b) regenerating a plant from the transformed plant cell, where the plant expresses the variant PEPC and has enhanced aluminum resistance as compared to a control plant of the same species that does not express the variant PEPC.

### V. Plants and Cultivation Thereof

Further aspects of the disclosure relate generally to plants comprising a variant PEPC described above, as well as methods of cultivating them.

Accordingly, in one aspect, the present disclosure provides a plant expressing a variant phosphoenolpyruvate carboxylase (PEPC), where the variant PEPC comprises at least one amino acid substitution at a position corresponding to one or more of residues A651, G678, A776, T778, and R886, in the consensus sequence of SEQ ID NO:8, where the plant was not grown from seeds subjected to ethyl methanesulfonate mutagenesis (EMS) mutagenesis, or the plant was not a progeny of an ancestral plant grown from seeds subjected to EMS mutagenesis, and where the amino acid sequence of the variant is at least 95% identical to the consensus sequence of SEQ ID NO:8. In some embodiments, the plant of the present disclosure comprises a variant PEPC that is expressed in roots of the plant. In some embodiments, the plant of the present disclosure has enhanced aluminum resistance as compared to a control plant of the same species that does not express the variant PEPC.

Any plant may be subjected to methods disclosed herein to express a variant PEPC of the present disclosure. In some embodiments, the plant is a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. In particular embodiments, the plant is maize (Zea mays). In some embodiments, the plant is soybean (Glycine max), wheat (Triticum aestivum), barley (Hordeum vulgare), rice (Oryza sativa), or sorghum (Sorghum bicolor).

In another aspect, the present disclosure provides a method of enhancing aluminum resistance in a plant, comprising: (a) crossing the plant of any one of the preceding embodiments with a second plant of the same genus or same species to generate F1 seeds; (b) growing F1 plants from the F1 seeds in a phytotoxic substrate, and (c) selecting a plant with enhanced aluminum resistance as compared to the second plant, where the phytotoxic substrate is an acidic substrate having a pH from 2-5 and micromolar or higher levels of Al³⁺.

In some embodiments, plants having a variant PEPC and enhanced aluminum resistance may be identified using available techniques in the art, e.g., visual stains for polysaccharide callose (an indication of aluminum-dependent damage) and visual stains for internalized aluminum (e.g., morin), as described in Example 2.

In some embodiments, the present disclosure relates to a part of the plant having enhanced aluminum resistance, where the plant part contains a variant PEPC of any of the preceding embodiments. In some embodiments, the plant part is a stem, a branch, a root, a leaf, a flower, a fruit, a seed, a cutting, a bud, a cell, or a portion thereof. In some embodiments, the present disclosure provides seed from which the plant can be grown.

### A. Carbon Sequestration in Soil

Release of organic acids into the root growth environment is a major contributor to deposition of carbon-based compounds into soils. Increased PEPC activity in roots increases production of organic acids including malate and pyruvate. In this way, increased release of organic acids by plants engineered to express the variant PEPC enzymes of the present disclosure increases sequestration of carbon into soil. In particular, plants engineered to express the variant PEPC enzymes of the present disclosure are contemplated to more effectively remove carbon dioxide from the atmosphere by more effectively depositing carbon-containing compounds into the soil, relative to a control plant of the same species (e.g., wild type or parental plant) that does not express the variant PEPC.

### B. Extraction of Phosphate from Soil

Release of organic acids including malate and citrate into the root growth environment is important for extracting anionic nutrients such as phosphate from the soil. The organic acids compete with phosphate and other anions for binding to cations such as aluminum and iron, thus releasing anions such as phosphate for uptake by plants. In this way, increased release of organic acids by plants engineered to express the variant PEPC enzymes of the present disclosure increases the capability of plants to extract nutrients from the soil, relative to a control plant of the same species (e.g., wild type or parental plant) that does not express the variant PEPC.

### C. Production of Essential Amino Acids

Oxaloacetate, the immediate product of PEPC, is the precursor to the amino acid aspartic acid. The amino acids asparagine, lysine, threonine, methionine, and isoleucine are all derived from aspartic acid. Lysine, threonine, methionine, and isoleucine are all considered to be essential nutrients for animals including humans. Hence, plants engineered to express the variant PEPC enzymes of the present disclosure are contemplated to produce higher levels of oxaloacetate and higher levels of aspartate-derived essential amino acids, relative to a control plant of the same species (e.g., wild type or parental plant) that does not express the variant PEPC.

### D. Production of Glucose

Oxaloacetate produced by PEPC is converted by malate dehydrogenase to malate, which through C4 photosynthesis supplies CO₂ for synthesis of glucose via the Calvin Cycle. C4 PEPC represents a unique variant of PEPC strictly related to photosynthesis in planta. Introduction of A651V, G678S, and T778I in the consensus sequence of SEQ ID NO:8 to maize C4 PEPC (ZmPPC1) each results in increased activity of C4 PEPC consistent with what was observed in the context of Arabidopsis C3 PEPC (AtPPC1). Hence, plants engineered to express the variant C4 PEPC enzymes of this disclosure are contemplated to produce higher levels of oxaloacetate, malate, and consequently glucose relative to a control plant of the same species (e.g. wild type or parental plant) that does not express the variant PEPC.

### EXEMPLARY EMBODIMENTS

1. A plant expressing a variant phosphoenolpyruvate carboxylase (PEPC), wherein the variant PEPC comprises:
(i) an amino acid substitution at a position corresponding to one or more of residues A651, G678, and T778, in the consensus sequence of SEQ ID NO:8, and/or
(ii) an amino acid substitution at a position corresponding to one or both of residue A776 and R886, in the consensus sequence of SEQ ID NO:8,

optionally wherein the plant was not grown from seeds subjected to ethyl methane sulfonate mutagenesis (EMS) mutagenesis, or the plant was not a progeny of an ancestral plant grown from seeds subjected to EMS mutagenesis, and/or

optionally wherein the amino acid sequence of the variant is:
(a) at least 90% identical to any one of SEQ ID NOs:1-9 and 15; or
(b) at least 95% identical to the consensus sequence of SEQ ID NO:8; or
(c) at least 95% identical to the consensus sequence of SEQ ID NO:9.

2. The plant of embodiment 1, wherein the variant PEPC comprises a further amino acid substitution at a position corresponding to one or more of residues R637, X675, K831, R890 and N965 in the consensus sequence of SEQ ID NO:8, wherein X675 is Q675 or H675.

3. The plant of embodiment 1, wherein the variant PEPC comprises an amino acid substitution at a position corresponding to one or more of residues A651, G678, and T778 in the consensus sequence of SEQ ID NO:8.

4. The plant of embodiment 3, wherein the variant PEPC further comprises an amino acid substitution at a position corresponding to one or both of A776 and R886 in the consensus sequence of SEQ ID NO:8.

5. The plant of embodiment 1, wherein the variant PEPC comprises one or more amino acid substitutions selected from the group consisting of A651V, G678S, A776S, T778I, and R886G.

6. The plant of embodiment 1, wherein the variant PEPC comprises one or more amino acid substitutions selected from the group consisting of A651V, G678S, and T778I.

7. The plant of embodiment 6, wherein the variant PEPC further comprises an amino acid substitution selected from the group consisting of one or both of A776S and R886G.

8. The plant of embodiment 1, wherein the amino acid sequence of the variant is at least 99% identical to SEQ ID NO:9, and the amino acid sequence of the variant does not consist of SEQ ID NO:10, SEQ ID NO:11, or SEQ ID NO:12.

9. The plant of embodiment 1, wherein the variant PEPC is expressed in roots of the plant.

10. The plant of any one of embodiments 1-9, wherein the plant has enhanced aluminum resistance as compared to a control plant of the same species that does not express the variant PEPC.

11. The plant of embodiment 10, wherein:
(a) growth of the plant is greater in a phytotoxic substrate as compared to the control plant when grown under the same conditions; and/or
(b) aluminum accumulation in roots of the plant is reduced after growth in the phytotoxic substrate as compared to the control plant when grown under the same conditions; and/or
(c) carbon-containing organic acid accumulation in the phytotoxic substrate is increased after growth of the plant in the phytotoxic substrate as compared to the control plant when grown under the same conditions,

wherein the phytotoxic substrate is a growth substrate having a pH from 2-5 and nanomolar or higher levels of Al³⁺.

12. The plant of any one of embodiments 1-11, wherein the plant is not Arabidopsis, optionally wherein the plant is selected from the group consisting of maize, soybean, wheat, barley, rice and sorghum, optionally wherein the plant is maize.

13. An isolated nucleic acid encoding a variant phosphoenolpyruvate carboxylase (PEPC) comprising:
(i) an amino acid substitution at a position corresponding to one or more of residues A651, G678, and T778, in the consensus sequence of SEQ ID NO:8, and/or
(ii) an amino acid substitution at a position corresponding to one or both of residue A776 and R886, in the consensus sequence of SEQ ID NO:8,

optionally wherein the amino acid sequence of the variant is:
(a) at least 90% identical to any one of SEQ ID NOs:1-9 and 15; or
(b) at least 95% identical to the consensus sequence of SEQ ID NO:8; or
(c) at least 95% identical to the consensus sequence of SEQ ID NO:9; and the amino acid sequence of the variant does not consist of SEQ ID NO:10, SEQ ID NO:11, or SEQ ID NO:12.

14. The nucleic acid of embodiment 13, wherein the variant PEPC comprises a further amino acid substitution at a position corresponding to one or more of residues R637, X675, K831, R890 and N965 in the consensus sequence of SEQ ID NO:8, wherein X675 is Q675 or H675.

15. The nucleic acid of embodiment 13, wherein the variant PEPC comprises an amino acid substitution at a position corresponding to one or more of residues A651, G678, and T778 in the consensus sequence of SEQ ID NO:8.

16. The nucleic acid of embodiment 15, wherein the variant PEPC further comprises an amino acid substitution at a position corresponding to one or both of A776 and R886 in the consensus sequence of SEQ ID NO:8.

17. The nucleic acid of embodiment 13, wherein the variant PEPC comprises one or more amino acid substitutions selected from the group consisting of A651V, G678S, A776S, T778I, and R886G.

18. The nucleic acid of embodiment 13, wherein the variant PEPC comprises one or more amino acid substitutions selected from the group consisting of A651V, G678S, and T778I.

19. The nucleic acid of embodiment 18, wherein the variant PEPC further comprises an amino acid substitution selected from the group consisting of one or both of A776S and R886G.

20. The nucleic acid of embodiment 13, wherein the amino acid sequence of the variant is at least 99% identical to SEQ ID NO:9.

21. An expression cassette comprising a promoter operably linked to the nucleic acid of any one of embodiments 13-20.

22. The expression cassette of embodiment 21, wherein the promoter is heterologous to the nucleic acid.

23. The expression cassette of 22, wherein the promoter is a root-specific promoter.

24. The expression cassette of embodiment 22, wherein the promoter is a constitutive promoter.

25. The expression cassette of embodiment 22, wherein the promoter is an inducible promoter.

26. An expression vector comprising the expression cassette of any one of embodiments 22 to 25.

27. A host cell comprising the expression cassette of any one of embodiments 22 to 25.

28. The cell of embodiment 27, wherein the host cell is a plant cell.

29. A transgenic plant comprising or regenerated from the cell of embodiment 28.

30. The transgenic plant of embodiment 29, wherein the plant expresses the variant PEPC.

31. The transgenic plant of embodiment 30, wherein the plant has enhanced aluminum resistance as compared to a control plant of the same species that does not express the variant PEPC.

32. A method for producing a plant expressing a variant phosphoenolpyruvate carboxylase (PEPC), comprising:
(a) introducing the expression cassette of any one of embodiments 21 to 25 into a plant cell to form a transformed plant cell; and
(b) regenerating a plant from the transformed plant cell,

wherein the plant expresses the variant PEPC and has enhanced aluminum resistance as compared to a control plant of the same species that does not express the variant PEPC.

33. A method for producing a plant expressing a variant phosphoenolpyruvate carboxylase (PEPC), comprising:
(a) introducing a clustered regularly interspaced short palindromic repeats (CRISPR) associated protein (Cas9) genome-editing system into a plant cell to form a transformed plant cell comprising the nucleic acid of any one of embodiments 13-20; and
(b) regenerating a plant from the transformed plant cell,

wherein the plant expresses the variant PEPC and has enhanced aluminum resistance as compared to a control plant of the same species that does not express the variant PEPC.

34. A plant produced by the method of embodiment 32 or embodiment 33.

35. A method of enhancing aluminum resistance in a plant, comprising:
(a) crossing the plant of any one of embodiments 1-12 with a second plant of the same genus or same species to generate F1 seeds;
(b) growing F1 plants from the F1 seeds in a phytotoxic substrate, and
(c) selecting a plant with enhanced aluminum resistance as compared to the second plant,

wherein the phytotoxic substrate is a growth substrate having a pH from 2-5 and nanomolar or higher levels of Al³⁺.

36. Seed from which the plant of any one of the preceding embodiments can be grown.

37. A method for sequestering carbon in soil, comprising:

growing the plant of any one of embodiments 1-12, 29-31 and 34 in soil under conditions effective for production of a carbon-containing organic acid by the plant and release of the organic acid from roots of the plant into the soil.

38. A method for extracting phosphate from soil, comprising:

growing the plant of any one of embodiments 1-12, 29-31 and 34 in soil under conditions effective for production of a carbon-containing organic acid by the plant and release of the organic acid from roots of the plant into the soil resulting in extraction of inorganic phosphate from the soil by the roots of the plant.

39. The method of embodiment 37 or embodiment 38, wherein the organic acid comprises one or more of pyruvate, malate and citrate.

40. A method for producing an oxaloacetate-derived amino acid, comprising:

growing the plant of any one of embodiments 1-12, 29-31 and 34 in soil under conditions effective for production of an oxaloacetate-derived amino acid by the plant.

41. The method of embodiment 40, wherein the oxaloacetate-derived amino acid comprises one or more of asparagine, lysine, threonine, methionine, and isoleucine.

42. A method for producing glucose, comprising:

growing the plant of any one of embodiments 1-12, 29-31 and 34 in soil in the presence of light and under conditions effective for production of glucose by the plant.

43. The method of any one of embodiments 37-42, wherein the soil has nanomolar or higher levels of Al³⁺.

### EXAMPLES

Abbreviations: AtPPC1 (Arabidopsis thaliana PPC1); EMS (ethyl methanesulfonate); PEP (phosphoenolpyruvate); PEPC (phosphoenolpyruvate carboxylase); wt (wild type); ZmPEP7 (Zea mays PEP7); and ZmPPC1 (Zea mays PPC1).

Although, the present disclosure has been described in some detail by way of illustration and example for purposes of clarity and understanding, it will be apparent to those skilled in the art that certain changes and modifications may be practiced. Therefore, the following synthetic and biological examples should not be construed as limiting the scope of the present disclosure, which is delineated by the appended claims.

### Example 1—Identification of Arabidopsis Thaliana Mutants

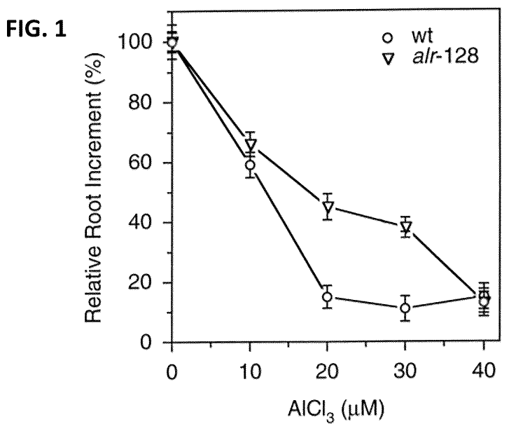

Three Arabidopsis thaliana mutants with increased aluminum resistance were isolated from a pool of ethyl methanesulfonate (EMS) mutagenized seeds (see, Larsen et al., Plant Physiol, 117: 9-18, 1998, herein incorporated by reference in its entirety). These mutants were identified by screening for those with greater than wild-type root growth in the presence of highly inhibitory levels of aluminum (FIG. 1). Through this screen, mutants with upwards of 4-fold higher growth in the presence of the highly inhibitory levels of aluminum were isolated. The isolated mutants were designated as alr-108, alr-128, and alr-139. All three mutants were found to possess amino acid substitutions in a phosphoenolpyruvate carboxylase (PEPC) sequence, relative to the amino acid sequence of wild type Arabidopsis thaliana PEPC set forth as SEQ ID NO:1 (UniProt ID NO. Q9MAHO). Mutant alr-108 contains the amino acid substitution A651V. Mutant alr-128 contains the amino acid substitution G678S. Mutant alr-139 contains the amino acid substitution T778I. Positions of substitutions are relative to the wild type Arabidopsis thaliana C3 PEPC sequence of SEQ ID NO:1 and the consensus sequence of SEQ ID NO:8.

### Example 2—Aluminum-Dependent Damage and Aluminum Accumulation

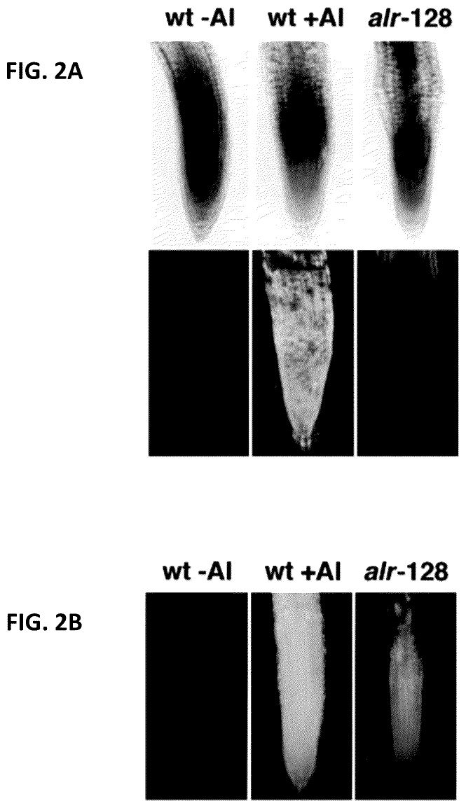

Subsequent to identification of the Arabidopsis mutants having enhanced aluminum-resistance, the physiological nature of the resistance was assessed. Mutant alr-128 was found to have reduced accumulation of the stress polysaccharide callose. In addition, this mutant was found to have reduced internalization of aluminum. Exemplary results are shown for mutant alr-128 (FIG. 2A and FIG. 2B). Thus, the aluminum-resistance phenotype was determined to be associated with enhanced aluminum exclusion in mutant alr-128.

### Example 3-Aluminum-Dependent Organic Acid Exudation

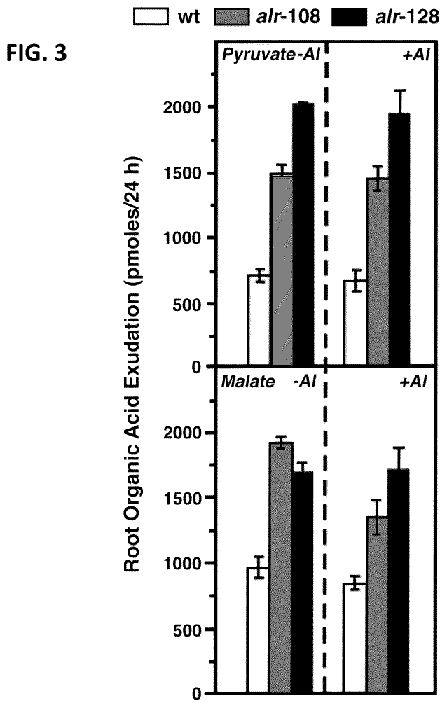

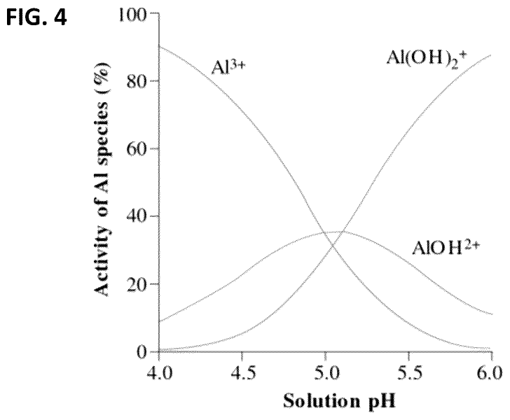

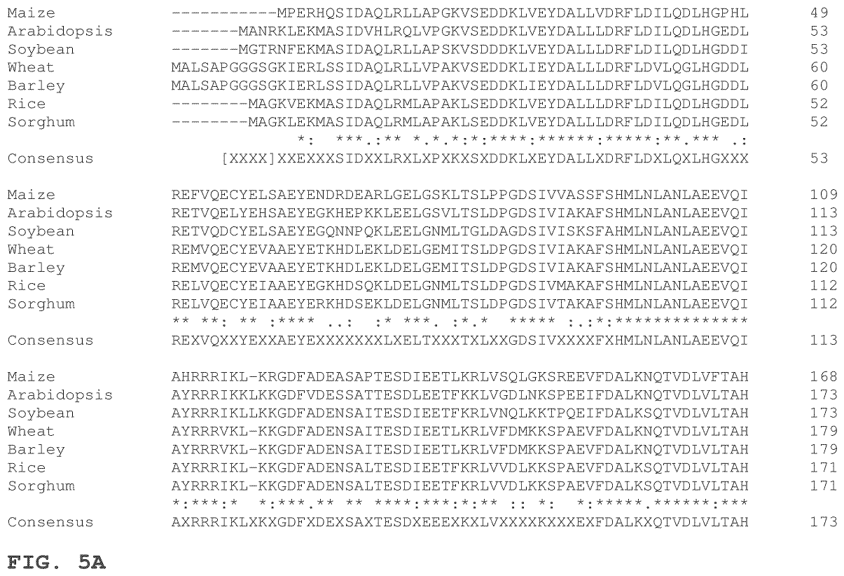

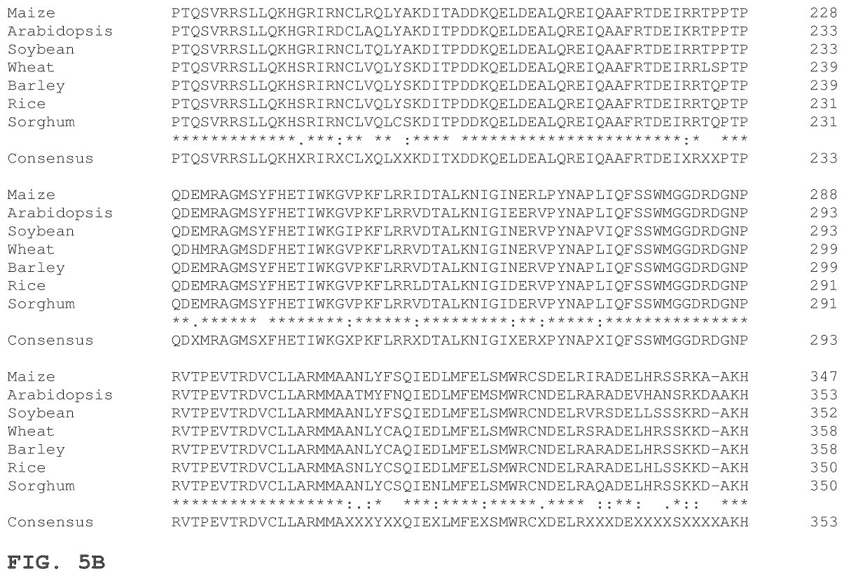

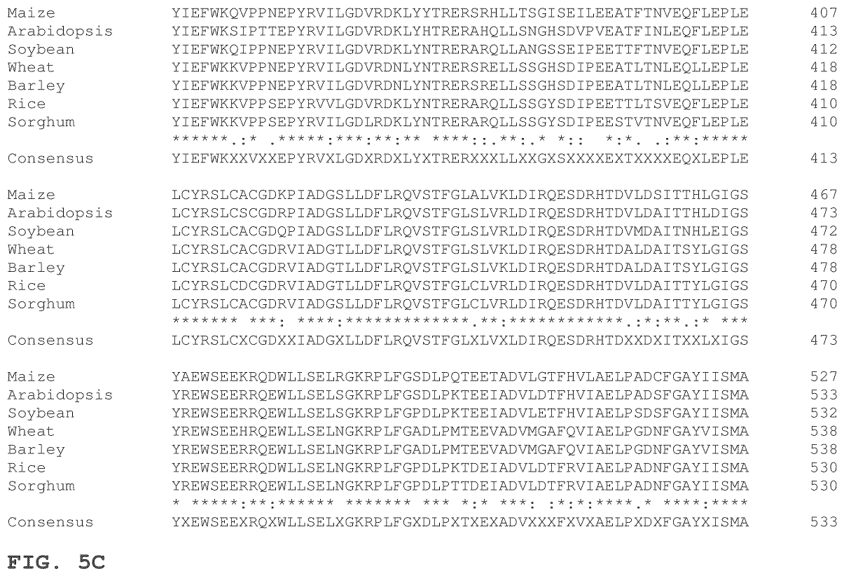

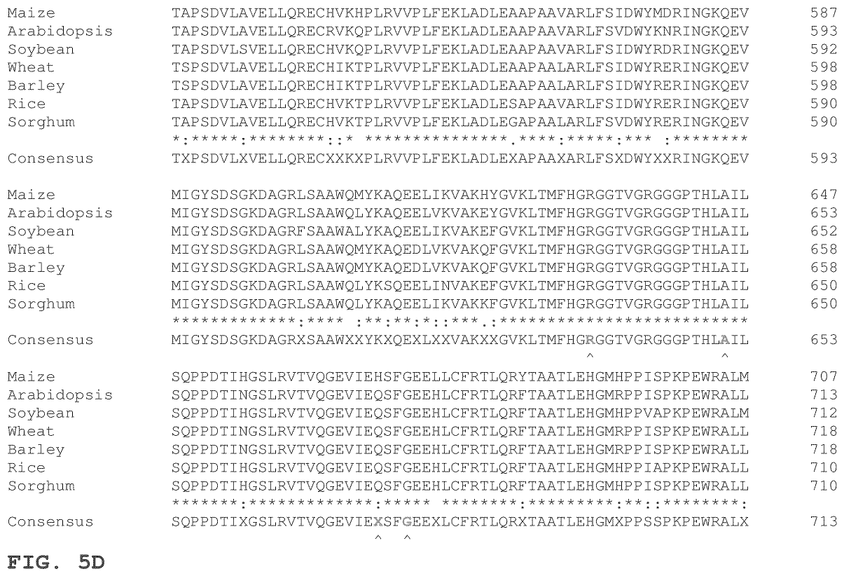

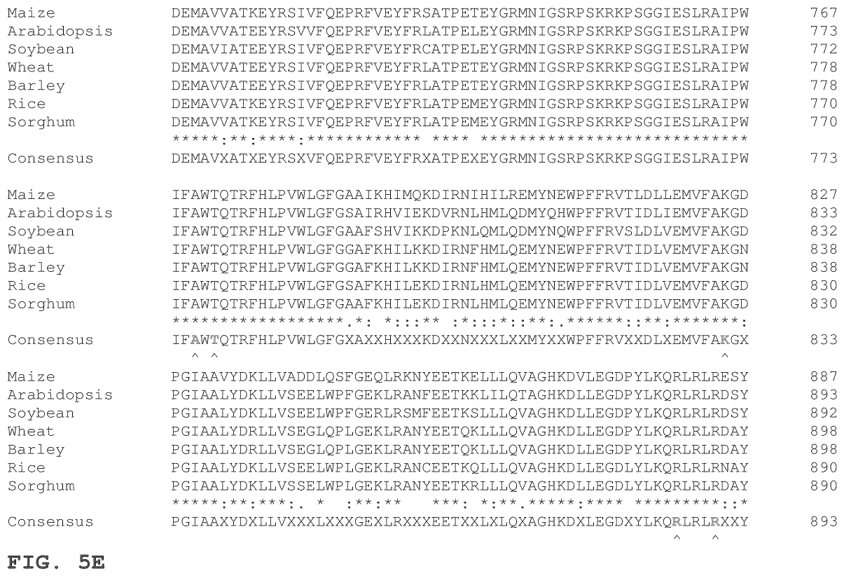

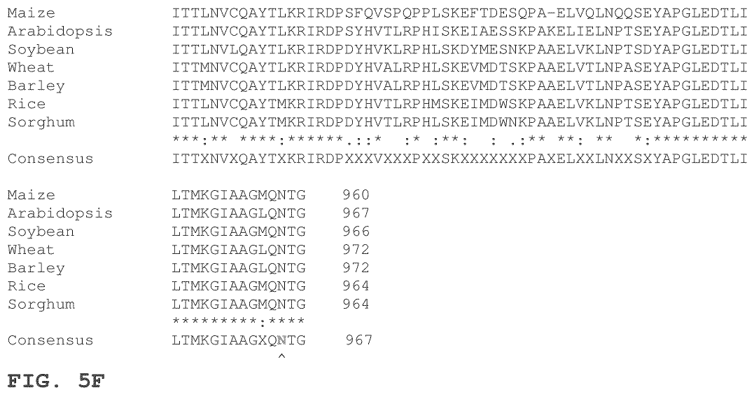

Further analysis of the mutants involved assessment of organic acid exudation. Mutants alr-108 and alr-128 were found to have increased levels of pyruvate and malate exudation, which was independent of the presence of aluminum (FIG. 3). Thus, the aluminum-resistance phenotype was determined to be associated with enhanced exudation of aluminum-chelating organic acids.

### Example 4—Analysis of Activity of Arabidopsis Thaliana and Zea Mays PEPC Enzymes

For both Arabidopsis C3 PPC1 (AtPPC1) and maize C4 PPC1 (ZmPPC1), the entire coding sequence for each was cloned into pET22b, which contains an amino-terminal 6x-HIS tag for protein purification, and expression was driven by the T7 promoter and lac operon. Relevant mutations were introduced by the Stratagene QuikChange mutagenesis kit using PCR amplification. Both wild type and mutant cDNA constructs were sequenced entirely following cloning into pet22b. Proteins were produced by growing transformed E. coli (BL21 CodonPlus) in autoinduction medium at 18° C. for 16 hours. Proteins were isolated by sonication of bacterial cells followed by passage over a nickel sepharose affinity column, elution with imidazole and separation on a GE SUPERDEX HILOAD 200 PG 16/60 all using an AKTA FPLC.

Enzyme analysis was conducted in vitro using protein that was generated by expression in E. coli using the pET22b vector. For expression, pET22b constructs were transformed into E. coli BL21-DE3 codon plus RIL cells and grown at 37° C. in autoinduction media until reaching an OD=1 after which these were transferred to 18° C. for 18 hours. Samples were collected and lysed by sonication. Protein was purified by passage through a His-trap nickel affinity column on an Akta FPLC and then eluted with 400 mM imidazole. Partially purified protein was then separated on an FPLC equipped with a HiPrep S200 size exclusion column (16/60) after which collected sample was concentrated by centrifugation to a final concentration of 4 mg/ml in 50% glycerol. Samples were stored at −20° C.

For enzymatic assays, a malate dehydrogenase coupled reaction was conducted with a range of substrate concentrations (0-7.5 mM phosphoenolpyruvate pyruvate) and a range of concentrations of allosteric inhibitor (0-50 mM malate). Assays were monitored using a Victor2 microplate reader at 25° C. Reactions consisted of 15 mM PPC enzyme variants, 50 mM HEPES pH 7.5, 10 mM MgCl₂, 10 mM KHCO₃ (carbonate substrate), 0.2 mM NADH, and 10 units/ml of malate dehydrogenase. Reaction time was 15 minutes and samples were measured on an ˜40 second interval. For measurement, which monitors loss of NADH, excitation wavelength was 340 nM and emission wavelength was 460 nM. From these assays, the enzyme kinetics of Tables 4-1 and 4-2 were determined for each enzyme variant.

TABLE 4-1
Arabidopsis thaliana and Engineered C3 PEPC Enzyme Kinetics

| A. thaliana | Kₘ PEP | Vₘₐₓ | | Kᵢ malate | |
|---|---|---|---|---|---|
| PPC1 | (mM) | (units/mg) | K_{cat}/Kₘ | (mM) | M* |
| AtPPC1 | 2.738 ± 0.166 | 24.992 ± 0.69 | 16.687 | 0.21 ± 0.02 | MX |
| wild type | | | | | |
| AtPPC1 | 1.111 ± 0.036 | 12.586 ± 0.14 | 20.769 | 17.43 ± 2.49 | CO |
| R886G | | | | | |
| AtPPC1 | 0.956 ± 0.040 | 15.776 ± 0.25 | 30.253 | 36.12 ± 8.71 | CO |
| A651V | | | | | |
| AtPPC1 | 0.433 ± 0.040 | 17.784 ± 0.44 | 75.300 | ND | MX |
| G678S | | | | | |
| AtPPC1 | 1.087 ± 0.027 | 14.738 ± 0.15 | 24.933 | 2.19 ± 0.21 | CO |
| T778I | | | | | |

*M = model of inhibition (MX—mixed, CO—competitive, NC—non-competitive)

TABLE 4-2
Zea mays and Engineered C4 PEPC Enzyme Kinetics

| Z. mays | Kₘ PEP | Vₘₐₓ | | Kᵢ malate | |
|---|---|---|---|---|---|
| PPC1 | (mM) | (units/mg) | K_{cat}/Kₘ | (mM) | M* |
| ZmPPC1 | 4.621 ± 0.406 | 17.56 ± 2.35 | 4.623 | 1.90 ± 0.08 | CO |
| wild type | | | | | |
| ZmPPC1 | 0.477 ± 0.042 | 12.84 ± 0.89 | 32.740 | 26.26 ± 3.80 | CO |
| A651V | | | | | |
| ZmPPC1 | 2.664 ± 0.333 | 17.74 ± 1.04 | 8.101 | 7.10 ± 0.43 | CO |
| G678S | | | | | |
| ZmPPC1 | 1.191 ± 0.104 | 14.05 ± 0.44 | 14.351 | 18.25 ± 1.24 | CO |
| T7781 | | | | | |

*M = model of inhibition (MX—mixed, CO—competitive, NC—non-competitive)

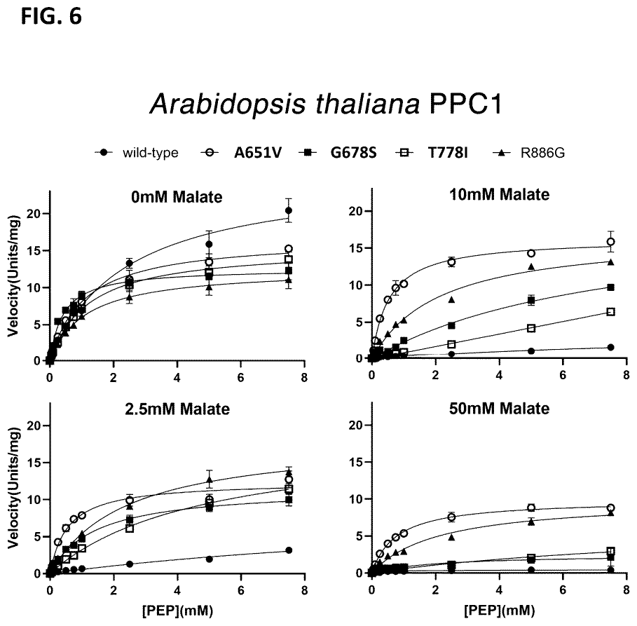

FIG. 6 and Table 4-1 show the enzymatic activity of wild type (wt) Arabidopsis thaliana C3 PPC1 (SEQ ID NO:1) as compared to engineered PEPC enzymes. An increase in K_{cat}/Kₘ, which represents catalytic efficiency, was observed for all AtPPC1 mutants relative to wt AtPPC1. The G678S (alr-128) mutant had the highest increase in catalytic efficiency, with a nearly 5-fold increase over wt AtPPC1. Additionally, the Kₘ, which is a measure of how tightly the enzyme binds to its PEP substrate (phosphoenolpyruvate), was found to be substantially decreased for all AtPPC1 mutants tested. Lower Kₘ represents a greater substrate-binding ability, even at low substrate concentrations. Comparison of wt and G678S AtPPC1 with regard to Kₘ showed that the G678S mutant has an 85% reduction in Kₘ and therefore binds substantially more tightly to the PEP substrate resulting in the large increase in catalytic efficiency.

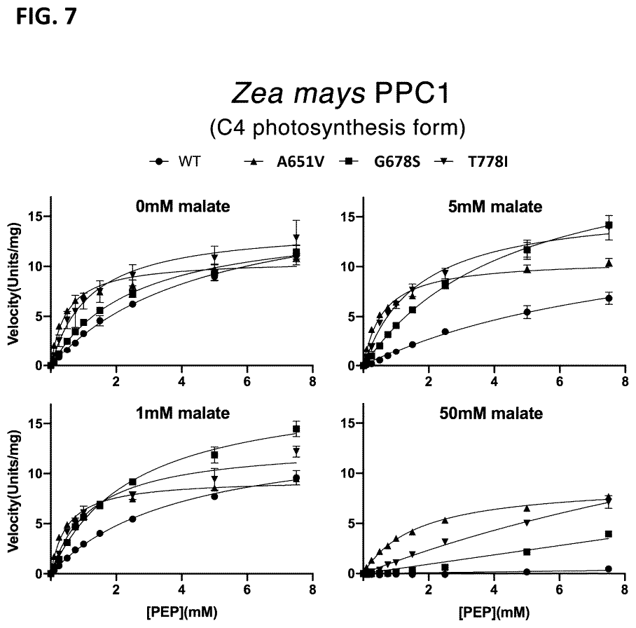

FIG. 7 and Table 4-2 show the enzymatic activity of wild type (wt) Zea mays C$ PPC1 (SEQ ID NO:15) as compared to engineered PEPC enzymes. In maize, ZmPPC1 is the enzyme responsible for C4 photosynthesis. ZmPP1 has serine (S) at position 776 and glycine (G) at position 886 relative to the consensus sequence of SEQ ID NO:8 (or said another way has S at position 780 and G at position 890 in SEQ ID NO:15). Throughout the present disclosure and unless indicated to the contrary, amino acid positions are numbered relative to SEQ ID NO:8 as determined when the amino acid sequence of an enzyme of interest is aligned to SEQ ID NO:8 using a pairwise alignment algorithm. In maize, all three of the mutations tested had universally positive effects on enzyme activity including reduced Kₘ (i.e. tighter binding to the substrate), greater catalytic efficiency, and substantially reduced effects of the allosteric inhibitor malate (Kᵢ). In particular, the A651V (alr-108) mutant had an increase in catalytic efficiency by upwards of 7-fold relative to wt ZmPPC1, and the G678S (alr-128) and T778I (alr-139) mutants had increases in catalytic efficiency by nearly 200% and 300% respectively, relative to wt ZmPPC1.

Malate is an allosteric inhibitor of both AtPPC1 and ZmPPC1, albeit in different manners. In the case of AtPPC1, malate inhibits in both a competitive and non-competitive way. Three of the amino acid changes (R886G, A651V and T778I) in AtPPC shift the model of inhibition to competitive. This indicates that these mutations unlink the malate binding pocket from the enzyme's active site, resulting in malate binding only directly affecting substrate affinity. In the case of ZmPPC1, the malate binding pocket for non-competitive inhibition is already compromised and is partially unlinked from the active site, thus the higher Kᵢ for wt ZmPPC1 compared to AtPPC1. All three aluminum-resistance mutations (A651V, G678S and T778I) in ZmPPC1 greatly reduce the effects of malate on enzyme function. In particular, wt ZmPPC1 is completely inhibited at 50 mM malate while the ZmPPC1 mutants are only partially affected or in the case of A651V ZmPPC1 wholly unaffected by malate.

### Example 5—Production of Transgenic Zea Mays

This example describes the production of transgenic maize engineered to express wild type and mutant PEPC enzymes.

For ZmPEP7 (C3 PEPC), a transgenic construct comprising the ZmPEP7 promoter along with the entirety of the ZmPEP7 genomic construct including 5′ and 3′-UTRs, all exons and introns from strain B104, was cloned into pDW3894. Alternatively, a root-specific promoter is used to drive expression of ZmPEP7 or variants thereof. pDW3894 is a T-DNA binary vector obtained from Iowa State University (Ames, IA). The nucleotide sequence of ZmPEP7 mRNA is set forth in NCBI No. NM_001112033, LOC542479, which corresponds to GeneID 542479. For maize transformation, wild type (ZmPEP7 amino acid sequence set forth in SEQ ID NO:2) and variant G672S in SEQ ID NO:2 (=G678S in SEQ ID NO:8) transgene constructs were generated. Constructs are transformed into Zea mays B104 germplasm via Agrobacterium-mediated transformation.

For ZmPPC1 (C4 PEPC), a transgenic construct comprising the ZmPPC1 promoter along with the entirety of the ZmPPC1 genomic construct including 5′ and 3′-UTRs, all exons and introns from strain B104, was cloned into pZY101. Alternatively, the ZmPEP7 promoter is used to drive expression of ZmPPC1 or variants thereof. pZY101 is a T-DNA binary vector purchased from Addgene (Watertown, MA). The nucleotide sequence ZmPPC1 mRNA is set forth in NCBI No. NM_001161348, LOC542372, which corresponds to GeneID 542372. For maize transformation, wild type (ZmPPC1 amino acid sequence set forth in SEQ ID NO:15) and variant A655V in SEQ ID NO:15 (=A651V in SEQ ID NO:8), G682S in SEQ ID NO: 15 (=G678S in SEQ ID NO:8), and T7821 in SEQ ID NO:15 (=T7781 in SEQ ID NO:8) transgene constructs were generated. Constructs are transformed into Zea mays B104 germplasm via Agrobacterium-mediated transformation.

### Example 6—Root Growth Analysis of Transgenic Arabidopsis Thaliana

Root growth was examined in transgenic Arabidopsis engineered to express wt AtPPC1, or a variant PEPC (G678S AtPPC1 or R886G AtPPC1) in a genotypic background devoid of expression of native AtPPC1. Seedlings from the transgenic Arabidopsis strains were grown at 20° C. in a soaked gel environment with aluminum toxicity equivalent to about 50-100 μM in the absence of agar. Root growth was assessed at day 7. A large increase in root growth was observed in the presence of aluminum in transgenic plants expressing G678S AtPPC1 or R886G AtPPC1. The increase in root growth in the presence of a phytotoxic substrate (high levels of aluminum) indicates that both the G678S AtPPC1 and the R886G AtPPC1 transgenic Arabidopsis have enhanced levels of aluminum resistance in comparison to plants expressing wt AtPPC1. This is consistent with the improved enzyme kinetics of the G678S AtPPC1 or R886G AtPPC1 enzymes described in Example 5.

SEQUENCES

>SEQ ID NO: l_Arabidopsis_thaliana (thale cress)
MANRKLEKMASIDVHLRQLVPGKVSEDDKLVEYDALLLDRFLDILQDLHGEDLRETVQELYEHS
AEYEGKHEPKKLEELGSVLTSLDPGDSIVIAKAFSHMLNLANLAEEVQIAYRRRIKKLKKGDFV
DESSATTESDLEETFKKLVGDLNKSPEEIFDALKNQTVDLVLTAHPTQSVRRSLLQKHGRIRDC
LAQLYAKDITPDDKQELDEALQREIQAAFRTDEIKRTPPTPQDEMRAGMSYFHETIWKGVPKFL
RRVDTALKNIGIEERVPYNAPLIQFSSWMGGDRDGNPRVTPEVTRDVCLLARMMAATMYFNQIE
DLMFEMSMWRCNDELRARADEVHANSRKDAAKHYIEFWKSIPTTEPYRVILGDVRDKLYHTRER
AHQLLSNGHSDVPVEATFINLEQFLEPLELCYRSLCSCGDRPIADGSLLDFLRQVSTFGLSLVR
LDIRQESDRHTDVLDAITTHLDIGSYREWSEERRQEWLLSELSGKRPLFGSDLPKTEEIADVLD
TFHVIAELPADSFGAYIISMATAPSDVLAVELLQRECRVKQPLRVVPLFEKLADLEAAPAAVAR
LFSVDWYKNRINGKQEVMIGYSDSGKDAGRLSAAWQLYKAQEELVKVAKEYGVKLTMFHGRGGT
VGRGGGPTHLAILSQPPDTINGSLRVTVQGEVIEQSFGEEHLCFRTLQRFTAATLEHGMRPPIS
PKPEWRALLDEMAVVATEEYRSVVFQEPRFVEYFRLATPELEYGRMNIGSRPSKRKPSGGIESL
RAIPWIFAWTQTRFHLPVWLGFGSAIRHVIEKDVRNLHMLQDMYQHWPFFRVTIDLIEMVFAKG
DPGIAALYDKLLVSEELWPFGEKLRANFEETKKLILQTAGHKDLLEGDPYLKQRLRLRDSYITT
LNVCQAYTLKRIRDPSYHVTLRPHISKEIAESSKPAKELIELNPTSEYAPGLEDTLILTMKGIA
AGLQNTG
>SEQ ID NO: 2_Zea_mays (maize PEP7, 03, root, anaplerosis)
MPERHQSIDAQLRLLAPGKVSEDDKLVEYDALLVDRFLDILQDLHGPHLREFVQECYELSAEYE
NDRDEARLGELGSKLTSLPPGDSIVVASSFSHMLNLANLAEEVQIAHRRRIKLKRGDFADEASA
PTESDIEETLKRLVSQLGKSREEVFDALKNQTVDLVFTAHPTQSVRRSLLQKHGRIRNCLRQLY
AKDITADDKQELDEALQREIQAAFRTDEIRRTPPTPQDEMRAGMSYFHETIWKGVPKFLRRIDT
ALKNIGINERLPYNAPLIQFSSWMGGDRDGNPRVTPEVTRDVCLLARMMAANLYFSQIEDLMFE
LSMWRCSDELRIRADELHRSSRKAAKHYIEFWKQVPPNEPYRVILGDVRDKLYYTRERSRHLLT
SGISEILEEATFTNVEQFLEPLELCYRSLCACGDKPIADGSLLDFLRQVSTFGLALVKLDIRQE
SDRHTDVLDSITTHLGIGSYAEWSEEKRQDWLLSELRGKRPLFGSDLPQTEETADVLGTFHVLA
ELPADCFGAYIISMATAPSDVLAVELLQRECHVKHPLRVVPLFEKLADLEAAPAAVARLFSIDW
YMDRINGKQEVMIGYSDSGKDAGRLSAAWQMYKAQEELIKVAKHYGVKLTMFHGRGGTVGRGGG
PTHLAILSQPPDTIHGSLRVTVQGEVIEHSFGEELLCFRTLQRYTAATLEHGMHPPISPKPEWR
ALMDEMAVVATKEYRSIVFQEPRFVEYFRSATPETEYGRMNIGSRPSKRKPSGGIESLRAIPWI
FAWTQTRFHLPVWLGFGAAIKHIMQKDIRNIHILREMYNEWPFFRVTLDLLEMVFAKGDPGIAA
VYDKLLVADDLQSFGEQLRKNYEETKELLLQVAGHKDVLEGDPYLKQRLRLRESYITTLNVCQA
YTLKRIRDPSFQVSPQPPLSKEFTDESQPAELVQLNQQSEYAPGLEDTLILTMKGIAAGMQNTG
In SEQ ID NO: 2: i) A at position 770 is a hallmark for 03 and
would be S if C4; and ii) R at position 880 is a hallmark for 03
and would be G if C4.
>SEQ ID NO: 3_Glycine_max (soybean)
MGTRNFEKMASIDAQLRLLAPSKVSDDDKLVEYDALLLDRFLDILQDLHGDDIRETVQDCYELS
AEYEGQNNPQKLEELGNMLTGLDAGDSIVISKSFAHMLNLANLAEEVQIAYRRRIKLLKKGDFA
DENSAITESDIEETFKRLVNQLKKTPQEIFDALKSQTVDLVLTAHPTQSVRRSLLQKHGRIRNC
LTQLYAKDITPDDKQELDEALQREIQAAFRTDEIRRTPPTPQDEMRAGMSYFHETIWKGIPKFL
RRVDTALKNIGINERVPYNAPVIQFSSWMGGDRDGNPRVTPEVTRDVCLLARMMAANLYFSQIE
DLMFELSMWRCNDELRVRSDELLSSSKRDAKHYIEFWKQIPPNEPYRVILGDVRDKLYNTRERA
RQLLANGSSEIPEETTFTNVEQFLEPLELCYRSLCACGDQPIADGSLLDFLRQVSTFGLSLVRL
DIRQESDRHTDVMDAITNHLEIGSYREWSEERRQEWLLSELSGKRPLFGPDLPKTEEIADVLET
FHVIAELPSDSFGAYIISMATAPSDVLSVELLQRECHVKQPLRVVPLFEKLADLEAAPAAVARL
FSIDWYRDRINGKQEVMIGYSDSGKDAGRFSAAWALYKAQEELIKVAKEFGVKLTMFHGRGGTV
GRGGGPTHLAILSQPPDTIHGSLRVTVQGEVIEQSFGEEHLCFRTLQRFTAATLEHGMHPPVAP
KPEWRALMDEMAVIATEEYRSIVFQEPRFVEYFRCATPELEYGRMNIGSRPSKRKPSGGIESLR
AIPWIFAWTQTRFHLPVWLGFGAAFSHVIKKDPKNLQMLQDMYNQWPFFRVSLDLVEMVFAKGD
PGIAALYDKLLVSEELWPFGERLRSMFEETKSLLLQVAGHKDLLEGDPYLKQRLRLRDSYITTL
NVLQAYTLKRIRDPDYHVKLRPHLSKDYMESNKPAAELVKLNPTSDYAPGLEDTLILTMKGIAA
GMQNTG
>SEQ ID NO: 4_Triticum_aestivum (wheat)
MALSAPGGGSGKIERLSSIDAQLRLLVPAKVSEDDKLIEYDALLLDRFLDVLQGLHGDDLREMV
QECYEVAAEYETKHDLEKLDELGEMITSLDPGDSIVIAKAFSHMLNLANLAEEVQIAYRRRVKL
KKGDFADENSAITESDIEETLKRLVFDMKKSPAEVFDALKNQTVDLVLTAHPTQSVRRSLLQKH
SRIRNCLVQLYSKDITPDDKQELDEALQREIQAAFRTDEIRRLSPTPQDHMRAGMSDFHETIWK
GVPKFLRRVDTALKNIGINERVPYNAPLIQFSSWMGGDRDGNPRVTPEVTRDVCLLARMMAANL
YCAQIEDLMFELSMWRCNDELRSRADELHRSSKKDAKHYIEFWKKVPPNEPYRVILGDVRDNLY
NTRERSRELLSSGHSDIPEEATLTNLEQLLEPLELCYRSLCACGDRVIADGTLLDFLRQVSTFG
LSLVKLDIRQESDRHTDALDAITSYLGIGSYREWSEEHRQEWLLSELNGKRPLFGADLPMTEEV
ADVMGAFQVIAELPGDNFGAYVISMATSPSDVLAVELLQRECHIKTPLRVVPLFEKLADLEAAP
AALARLFSIDWYRERINGKQEVMIGYSDSGKDAGRLSAAWQMYKAQEDLVKVAKQFGVKLTMFH
GRGGTVGRGGGPTHLAILSQPPDTINGSLRVTVQGEVIEQSFGEEHLCFRTLQRFTAATLEHGM
RPPISPKPEWRALLDEMAVVATEEYRSIVFQEPRFVEYFRLATPETEYGRMNIGSRPSKRKPSG
GIESLRAIPWIFAWTQTRFHLPVWLGFGGAFKHILKKDIRNFHMLQEMYNEWPFFRVTIDLVEM
VFAKGNPGIAALYDRLLVSEGLQPLGEKLRANYEETQKLLLQVAGHKDLLEGDPYLKQRLRLRD
AYITTMNVCQAYTLKRIRDPDYHVALRPHLSKEVMDTSKPAAELVTLNPASEYAPGLEDTLILT
MKGIAAGLQNTG
>SEQ ID NO: 5_Hordeum vulgare (barley)
MALSAPGGGSGKIERLSSIDAQLRLLVPAKVSEDDKLIEYDALLLDRFLDVLQGLHGDDLREMV
QECYEVAAEYETKHDLEKLDELGEMITSLDPGDSIVIAKAFSHMLNLANLAEEVQIAYRRRVKL
KKGDFADENSAITESDIEETLKRLVFDMKKSPAEVFDALKNQTVDLVLTAHPTQSVRRSLLQKH
SRIRNCLVQLYSKDITPDDKQELDEALQREIQAAFRTDEIRRTQPTPQDEMRAGMSYFHETIWK
GVPKFLRRVDTALKNIGINERVPYNAPLIQFSSWMGGDRDGNPRVTPEVTRDVCLLARMMAANL
YCAQIEDLMFELSMWRCNDELRARADELHRSSKKDAKHYIEFWKKVPPNEPYRVILGDVRDNLY
NTRERSRELLSSGHSDIPEEATLTNLEQLLEPLELCYRSLCACGDRVIADGTLLDFLRQVSTFG
LSLVKLDIRQESDRHTDALDAITSYLGIGSYREWSEERRQEWLLSELNGKRPLFGADLPMTEEV
ADVMGAFQVIAELPGDNFGAYVISMATSPSDVLAVELLQRECHIKTPLRVVPLFEKLADLEAAP
AALARLFSIDWYRERINGKQEVMIGYSDSGKDAGRLSAAWQMYKAQEDLVKVAKQFGVKLTMFH
GRGGTVGRGGGPTHLAILSQPPDTINGSLRVTVQGEVIEQSFGEEHLCFRTLQRFTAATLEHGM
RPPISPKPEWRALLDEMAVVATEEYRSIVFQEPRFVEYFRLATPETEYGRMNIGSRPSKRKPSG
GIESLRAIPWIFAWTQTRFHLPVWLGFGGAFKHILKKDIRNFHMLQEMYNEWPFFRVTIDLVEM
VFAKGNPGIAALYDRLLVSEGLQPLGEKLRANYEETQKLLLQVAGHKDLLEGDPYLKQRLRLRD
AYITTMNVCQAYTLKRIRDPDYHVALRPHLSKEVMDTSKPAAELVTLNPASEYAPGLEDTLILT
MKGIAAGLQNTG
>SEQ ID NO: 6_Oryza_sativa (rice)
MAGKVEKMASIDAQLRMLAPAKLSEDDKLVEYDALLLDRFLDILQDLHGDDLRELVQECYEIAA
EYEGKHDSQKLDELGNMLTSLDPGDSIVMAKAFSHMLNLANLAEEVQIAYRRRIKLKKGDFADE
NSALTESDIEETFKRLVVDLKKSPAEVFDALKSQTVDLVLTAHPTQSVRRSLLQKHSRIRNCLV
QLYSKDITPDDKQELDEALQREIQAAFRTDEIRRTQPTPQDEMRAGMSYFHETIWKGVPKFLRR
LDTALKNIGIDERVPYNAPLIQFSSWMGGDRDGNPRVTPEVTRDVCLLARMMASNLYCSQIEDL
MFELSMWRCNDELRARADELHLSSKKDAKHYIEFWKKVPPSEPYRVVLGDVRDKLYNTRERARQ
LLSSGYSDIPEETTLTSVEQFLEPLELCYRSLCDCGDRVIADGTLLDFLRQVSTFGLCLVRLDI
RQESDRHTDVLDAITTYLGIGSYREWSEERRQDWLLSELNGKRPLFGPDLPKTDEIADVLDTFR
VIAELPADNFGAYIISMATAPSDVLAVELLQRECHVKTPLRVVPLFEKLADLESAPAAVARLFS
IDWYRERINGKQEVMIGYSDSGKDAGRLSAAWQLYKSQEELINVAKEFGVKLTMFHGRGGTVGR
GGGPTHLAILSQPPDTIHGSLRVTVQGEVIEQSFGEEHLCFRTLQRFTAATLEHGMHPPIAPKP
EWRALLDEMAVVATKEYRSIVFQEPRFVEYFRLATPEMEYGRMNIGSRPSKRKPSGGIESLRAI
PWIFAWTQTRFHLPVWLGFGSAFKHILEKDIRNLHMLQEMYNEWPFFRVTIDLVEMVFAKGDPG
IAALYDKLLVSEELWPLGEKLRANCEETKQLLLQVAGHKDLLEGDLYLKQRLRLRNAYITTLNV
CQAYTMKRIRDPDYHVTLRPHMSKEIMDWSKPAAELVKLNPTSEYAPGLEDTLILTMKGIAAGM
QNTG
>SEQ ID NO: 7_Sorghum_bicolor (broomcorn)
MAGKLEKMASIDAQLRMLAPAKLSEDDKLVEYDALLLDRFLDILQDLHGEDLRELVQECYEIAA
EYERKHDSEKLDELGNMLTSLDPGDSIVTAKAFSHMLNLANLAEEVQIAYRRRIKLKKGDFADE
NSALTESDIEETFKRLVVDLKKSPAEVFDALKSQTVDLVLTAHPTQSVRRSLLQKHSRIRNCLV
QLCSKDITPDDKQELDEALQREIQAAFRTDEIRRTQPTPQDEMRAGMSYFHETIWKGVPKFLRR
VDTALKNIGIDERVPYNAPLIQFSSWMGGDRDGNPRVTPEVTRDVCLLARMMAANLYCSQIENL
MFELSMWRCNDELRAQADELHRSSKKDAKHYIEFWKKVPPSEPYRVILGDLRDKLYNTRERARQ
LLSSGYSDIPEESTVTNVEQFLEPLELCYRSLCACGDRVIADGSLLDFLRQVSTFGLCLVRLDI
RQESDRHTDVLDAITTYLGIGSYREWSEERRQEWLLSELNGKRPLFGPDLPTTDEIADVLDTFR
VIAELPADNFGAYIISMATAPSDVLAVELLQRECHVKTPLRVVPLFEKLADLEGAPAALARLFS
VDWYRERINGKQEVMIGYSDSGKDAGRLSAAWQLYKAQEELIKVAKKFGVKLTMFHGRGGTVGR
GGGPTHLAILSQPPDTIHGSLRVTVQGEVIEQSFGEEHLCFRTLQRFTAATLEHGMHPPISPKP
EWRALLDEMAVVATKEYRSIVFQEPRFVEYFRLATPEMEYGRMNIGSRPSKRKPSGGIESLRAI
PWIFAWTQTRFHLPVWLGFGAAFKHILEKDIRNLHMLQEMYNEWPFFRVTIDLVEMVFAKGDPG
IAALYDKLLVSSELWPLGEKLRANYEETKRLLLQVAGHKDLLEGDLYLKQRLRLRDAYITTLNV
CQAYTMKRIRDPDYHVTLRPHLSKEIMDWNKPAAELVKLNPTSEYAPGLEDTLILTMKGIAAGM
QNTG
>SEQ ID NO: 8_Consensus
[XXXX]XXEXXXSIDXXLRXLXPXKXSXDDKLXEYDALLXDRFLDXLQXLHGXXX
REXVQXXYEXXAEYEXXXXXXXLXELTXXXTXLXXGDSIVXXXXFXHMLNLANLAEEVQI
AXRRRIKLXKXGDFXDEXSAXTESDXEEEXKXLVXXXXKXXXEXFDALKXQTVDLVLTAH
PTQSVRRSLLQKHXRIRXCLXQLXXKDITXDDKQELDEALQREIQAAFRTDEIXRXXPTP
QDXMRAGMSXFHETIWKGXPKFLRRXDTALKNIGIXERXPYNAPXIQFSSWMGGDRDGNP
RVTPEVTRDVCLLARMMAXXXYXXQIEXLMFEXSMWRCXDELRXXXDEXXXXSXXXXAKH
YIEFWKXXVXXEPYRVXLGDXRDXLYXTRERXXXLLXXGXSXXXXEXTXXXXEQXLEPLE
LCYRSLCXCGDXXIADGXLLDFLRQVSTFGLXLVXLDIRQESDRHTDXXDXITXXLXIGS
YXEWSEEXRQXWLLSELXGKRPLFGXDLPXTXEXADVXXXFXVXAELPXDXFGAYXISMA
TXPSDVLXVELLQRECXXKXPLRVVPLFEKLADLEXAPAAXARLFSXDWYXXRINGKQEV
MIGYSDSGKDAGRXSAAWXXYKXQEXLXXVAKXXGVKLTMFHGRGGTVGXGGGPTHLAIL
SQPPDTIXGSLRVTVQGEVIEXSFGEEXLCFRTLQRXTAATLEHGMXPPSSPKPEWRALX
DEMAVXATXEYRSXVFQEPRFVEYFRXATPEXEYGRMNIGSRPSKRKPSGGIESLRAIPW
IFAWTQTRFHLPVWLGFGXAXXHXXXKDXXNXXXLXXMYXXWPFFRVXXDLXEMVFAKGX
PGIAAXYDXLLVXXXLXXXGEXLRXXXEETXXLXLQXAGHKDXLEGDXYLKQRLRLRXXY
ITTXNVXQAYTXKRIRDPXXXVXXXPXXSKXXXXXXXPAXELXXLNXXSXYAPGLEDTLI
LTMKGIAAGXQNTG, wherein X at position 1, 2, 3 or 4 can be any
amino acid or absent.
>SEQ ID NO: 9_Refined_Consensus
[XXXX]XXE[K/R]XXSID[A/V][Q/H]LRXL[V/A]PXK[V/L]S[E/D]DDKL[V/I]EYD
ALL[L/V]DRFLD[I/V]LQ[D/G]LHGX[D/H][L/I]REXVQ[E/D][C/L]YEX[A/S]AE
YEXXXXXX[K/R]LXELGXX[L/I]T[S/G]L[D/P][P/A]GDSIVX[A/S][K/S][A/S]F
[S/A]HMLNLANLAEEVQIA[Y/H]RRRIKLXK[K/R]GDF[A/V]DEXSAXTESD[I/L]EET
[F/L]K[R/K]LVX[D/Q][L/M]XK[S/T][P/R]XE[V/I]FDALK[N/S]QTVDLV[L/F]
TAHPTQSVRRSLLQKH[S/G]RIRXCLXQL[Y/C][S/A]KDIT[P/A]DDKQELDEALQREIQ
AAFRTDEI[R/K]R[T/L]XPTPQD[E/H]MRAGMS[Y/D]FHETIWKG[V/I]PKFLRR[V/I]
DTALKNIGI[N/D]ERXPYNAP[L/V]IQFSSWMGGDRDGNPRVTPEVTRDVCLLARMMA[A/
S][N/T][L/M]Y[C/F]XQIE[D/N]LMFE[L/M]SMWRC[N/S]DELRX[R/Q][A/S]DE[
L/V][H/L]X[S/N]S[K/R][K/R][D/A]XAKHYIEFWKX[V/I]P[P/T]XEPYRV[I/V]
LGD[V/L]RD[K/N]LYXTRER[A/S][R/H]XLLX[S/N]GXS[D/E][I/V][P/L][E/V]
EXTX[T/I][N/S][V/L]EQ[F/L]LEPLELCYRSLCXCGDX[P/V]IADG[S/T]LLDFLRQ
VSTFGLXLV[R/K]LDIRQESDRHTD[V/A][L/M]D[S/A]ITX[Y/H]LXIGSY[R/A]EWS
EEXRQ[D/E]WLLSELXGKRPLFGXDLPXT[E/D]EXADV[L/M]X[T/A]FXV[I/L]AELPX
DXFGAY[I/V]ISMAT[A/S]PSDVL[A/S]VELLQREC[H/R][V/I]KXPLRVVPLFEKLAD
LE[A/G]APAA[L/V]ARLFS[I/V]DWYXXRINGKQEVMIGYSDSGKDAGR[L/F]SAAW[Q/
A][L/M]YK[S/A]QE[E/D]L[I/V][K/N]VAKX[F/Y]GVKLTMFHGRGGTVGRGGGPTHL
AILSQPPDTI[H/N]GSLRVTVQGEVIE[Q/H]SFGEE[H/L]LCFRTLQR[F/Y]TAATLEHG
MXPP[I/V][S/A]PKPEWRAL[L/M]DEMAV[V/I]AT[E/K]EYRS[I/V]VFQEPRFVEYF
RXATPEXEYGRMNIGSRPSKRKPSGGIESLRAIPWIFAWTQTRFHLPVWLGFGXA[F/I]XH[I/
V]XXKDX[R/K]NX[H/Q][I/M]L[Q/R][E/D]MY[N/Q]XWPFFRV[T/S][I/L]DLXE
MVFAKG[D/N]PGIAA[L/V]YD[K/R]LLV[S/A]XXL[W/Q][P/S][L/F]GEXLRX[N/M]
XEET[K/Q]XL[L/I]LQ[V/T]AGHKD[L/D]LEGD[P/L]YLKQRLRLR[D/E][A/S]YI
TT[L/M]NV[C/L]QAYT[L/M]KRIRDP[D/S][Y/F][H/Q]]VX[L/P][R/Q]P[H/P]X
SK[E/D]XX[D/E]X[S/N][K/Q]]PAXEL[V/I]XLN[P/Q]XS[E/D]YAPGLEDTLILTM
KGIAAG[M/L]QNTG, wherein X at position 1, 2, 3 or 4 can be any
amino acid or absent.
SEQ ID NO: 10_(At WITH A651V SUBSTITUTION)
MANRKLEKMASIDVHLRQLVPGKVSEDDKLVEYDALLLDRFLDILQDLHGEDLRETVQELYEHS
AEYEGKHEPKKLEELGSVLTSLDPGDSIVIAKAFSHMLNLANLAEEVQIAYRRRIKKLKKGDFV
DESSATTESDLEETFKKLVGDLNKSPEEIFDALKNQTVDLVLTAHPTQSVRRSLLQKHGRIRDC
LAQLYAKDITPDDKQELDEALQREIQAAFRTDEIKRTPPTPQDEMRAGMSYFHETIWKGVPKFL
RRVDTALKNIGIEERVPYNAPLIQFSSWMGGDRDGNPRVTPEVTRDVCLLARMMAATMYFNQIE
DLMFEMSMWRCNDELRARADEVHANSRKDAAKHYIEFWKSIPTTEPYRVILGDVRDKLYHTRER
AHQLLSNGHSDVPVEATFINLEQFLEPLELCYRSLCSCGDRPIADGSLLDFLRQVSTFGLSLVR
LDIRQESDRHTDVLDAITTHLDIGSYREWSEERRQEWLLSELSGKRPLFGSDLPKTEEIADVLD
TFHVIAELPADSFGAYIISMATAPSDVLAVELLQRECRVKQPLRVVPLFEKLADLEAAPAAVAR
LFSVDWYKNRINGKQEVMIGYSDSGKDAGRLSAAWQLYKAQEELVKVAKEYGVKLTMFHGRGGT
VGRGGGPTHLVILSQPPDTINGSLRVTVQGEVIEQSFGEEHLCFRTLQRFTAATLEHGMRPPIS
PKPEWRALLDEMAVVATEEYRSVVFQEPRFVEYFRLATPELEYGRMNIGSRPSKRKPSGGIESL
RAIPWIFAWTQTRFHLPVWLGFGSAIRHVIEKDVRNLHMLQDMYQHWPFFRVTIDLIEMVFAKG
DPGIAALYDKLLVSEELWPFGEKLRANFEETKKLILQTAGHKDLLEGDPYLKQRLRLRDSYITT
LNVCQAYTLKRIRDPSYHVTLRPHISKEIAESSKPAKELIELNPTSEYAPGLEDTLILTMKGIA
AGLQNTG
SEQ ID NO: 11_(At WITH G678S SUBSTITUTIONS)
MANRKLEKMASIDVHLRQLVPGKVSEDDKLVEYDALLLDRFLDILQDLHGEDLRETVQELYEHS
AEYEGKHEPKKLEELGSVLTSLDPGDSIVIAKAFSHMLNLANLAEEVQIAYRRRIKKLKKGDFV
DESSATTESDLEETFKKLVGDLNKSPEEIFDALKNQTVDLVLTAHPTQSVRRSLLQKHGRIRDC
LAQLYAKDITPDDKQELDEALQREIQAAFRTDEIKRTPPTPQDEMRAGMSYFHETIWKGVPKFL
RRVDTALKNIGIEERVPYNAPLIQFSSWMGGDRDGNPRVTPEVTRDVCLLARMMAATMYFNQIE
DLMFEMSMWRCNDELRARADEVHANSRKDAAKHYIEFWKSIPTTEPYRVILGDVRDKLYHTRER
AHQLLSNGHSDVPVEATFINLEQFLEPLELCYRSLCSCGDRPIADGSLLDFLRQVSTFGLSLVR
LDIRQESDRHTDVLDAITTHLDIGSYREWSEERRQEWLLSELSGKRPLFGSDLPKTEEIADVLD
TFHVIAELPADSFGAYIISMATAPSDVLAVELLQRECRVKQPLRVVPLFEKLADLEAAPAAVAR
LFSVDWYKNRINGKQEVMIGYSDSGKDAGRLSAAWQLYKAQEELVKVAKEYGVKLTMFHGRGGT
VGRGGGPTHLAILSQPPDTINGSLRVTVQGEVIEQSFSEEHLCFRTLQRFTAATLEHGMRPPIS
PKPEWRALLDEMAVVATEEYRSVVFQEPRFVEYFRLATPELEYGRMNIGSRPSKRKPSGGIESL
RAIPWIFAWTQTRFHLPVWLGFGSAIRHVIEKDVRNLHMLQDMYQHWPFFRVTIDLIEMVFAKG
DPGIAALYDKLLVSEELWPFGEKLRANFEETKKLILQTAGHKDLLEGDPYLKQRLRLRDSYITT
LNVCQAYTLKRIRDPSYHVTLRPHISKEIAESSKPAKELIELNPTSEYAPGLEDTLILTMKGIA
AGLQNTG
SEQ ID NO: 12_(At WITH T778I SUBSTITUTION)
MANRKLEKMASIDVHLRQLVPGKVSEDDKLVEYDALLLDRFLDILQDLHGEDLRETVQELYEHS
AEYEGKHEPKKLEELGSVLTSLDPGDSIVIAKAFSHMLNLANLAEEVQIAYRRRIKKLKKGDFV
DESSATTESDLEETFKKLVGDLNKSPEEIFDALKNQTVDLVLTAHPTQSVRRSLLQKHGRIRDC
LAQLYAKDITPDDKQELDEALQREIQAAFRTDEIKRTPPTPQDEMRAGMSYFHETIWKGVPKFL
RRVDTALKNIGIEERVPYNAPLIQFSSWMGGDRDGNPRVTPEVTRDVCLLARMMAATMYFNQIE
DLMFEMSMWRCNDELRARADEVHANSRKDAAKHYIEFWKSIPTTEPYRVILGDVRDKLYHTRER
AHQLLSNGHSDVPVEATFINLEQFLEPLELCYRSLCSCGDRPIADGSLLDFLRQVSTFGLSLVR
LDIRQESDRHTDVLDAITTHLDIGSYREWSEERRQEWLLSELSGKRPLFGSDLPKTEEIADVLD
TFHVIAELPADSFGAYIISMATAPSDVLAVELLQRECRVKQPLRVVPLFEKLADLEAAPAAVAR
LFSVDWYKNRINGKQEVMIGYSDSGKDAGRLSAAWQLYKAQEELVKVAKEYGVKLTMFHGRGGT
VGRGGGPTHLAILSQPPDTINGSLRVTVQGEVIEQSFGEEHLCFRTLQRFTAATLEHGMRPPIS
PKPEWRALLDEMAVVATEEYRSVVFQEPRFVEYFRLATPELEYGRMNIGSRPSKRKPSGGIESL
RAIPWIFAWIQTRFHLPVWLGFGSAIRHVIEKDVRNLHMLQDMYQHWPFFRVTIDLIEMVFAKG
DPGIAALYDKLLVSEELWPFGEKLRANFEETKKLILQTAGHKDLLEGDPYLKQRLRLRDSYITT
LNVCQAYTLKRIRDPSYHVTLRPHISKEIAESSKPAKELIELNPTSEYAPGLEDTLILTMKGIA
AGLQNTG
SEQ ID NO: 13_(At WITH A651V, G678S, & R886G SUBSTITUTIONS)
MANRKLEKMASIDVHLRQLVPGKVSEDDKLVEYDALLLDRFLDILQDLHGEDLRETVQELYEHS
AEYEGKHEPKKLEELGSVLTSLDPGDSIVIAKAFSHMLNLANLAEEVQIAYRRRIKKLKKGDFV
DESSATTESDLEETFKKLVGDLNKSPEEIFDALKNQTVDLVLTAHPTQSVRRSLLQKHGRIRDC
LAQLYAKDITPDDKQELDEALQREIQAAFRTDEIKRTPPTPQDEMRAGMSYFHETIWKGVPKFL
RRVDTALKNIGIEERVPYNAPLIQFSSWMGGDRDGNPRVTPEVTRDVCLLARMMAATMYFNQIE
DLMFEMSMWRCNDELRARADEVHANSRKDAAKHYIEFWKSIPTTEPYRVILGDVRDKLYHTRER
AHQLLSNGHSDVPVEATFINLEQFLEPLELCYRSLCSCGDRPIADGSLLDFLRQVSTFGLSLVR
LDIRQESDRHTDVLDAITTHLDIGSYREWSEERRQEWLLSELSGKRPLFGSDLPKTEEIADVLD
TFHVIAELPADSFGAYIISMATAPSDVLAVELLQRECRVKQPLRVVPLFEKLADLEAAPAAVAR
LFSVDWYKNRINGKQEVMIGYSDSGKDAGRLSAAWQLYKAQEELVKVAKEYGVKLTMFHGRGGT
VGRGGGPTHLVILSQPPDTINGSLRVTVQGEVIEQSFSEEHLCFRTLQRFTAATLEHGMRPPIS
PKPEWRALLDEMAVVATEEYRSVVFQEPRFVEYFRLATPELEYGRMNIGSRPSKRKPSGGIESL
RAIPWIFAWTQTRFHLPVWLGFGSAIRHVIEKDVRNLHMLQDMYQHWPFFRVTIDLIEMVFAKG
DPGIAALYDKLLVSEELWPFGEKLRANFEETKKLILQTAGHKDLLEGDPYLKQGLRLRDSYITT
LNVCQAYTLKRIRDPSYHVTLRPHISKEIAESSKPAKELIELNPTSEYAPGLEDTLILTMKGIA
AGLQNTG
SEQ ID NO: 14_(At WITH A651V, G678S, T778I & R886G SUBSTITUTIONS)
MANRKLEKMASIDVHLRQLVPGKVSEDDKLVEYDALLLDRFLDILQDLHGEDLRETVQELYEHS
AEYEGKHEPKKLEELGSVLTSLDPGDSIVIAKAFSHMLNLANLAEEVQIAYRRRIKKLKKGDFV
DESSATTESDLEETFKKLVGDLNKSPEEIFDALKNQTVDLVLTAHPTQSVRRSLLQKHGRIRDC
LAQLYAKDITPDDKQELDEALQREIQAAFRTDEIKRTPPTPQDEMRAGMSYFHETIWKGVPKFL
RRVDTALKNIGIEERVPYNAPLIQFSSWMGGDRDGNPRVTPEVTRDVCLLARMMAATMYFNQIE
DLMFEMSMWRCNDELRARADEVHANSRKDAAKHYIEFWKSIPTTEPYRVILGDVRDKLYHTRER
AHQLLSNGHSDVPVEATFINLEQFLEPLELCYRSLCSCGDRPIADGSLLDFLRQVSTFGLSLVR
LDIRQESDRHTDVLDAITTHLDIGSYREWSEERRQEWLLSELSGKRPLFGSDLPKTEEIADVLD
TFHVIAELPADSFGAYIISMATAPSDVLAVELLQRECRVKQPLRVVPLFEKLADLEAAPAAVAR
LFSVDWYKNRINGKQEVMIGYSDSGKDAGRLSAAWQLYKAQEELVKVAKEYGVKLTMFHGRGGT
VGRGGGPTHLVILSQPPDTINGSLRVTVQGEVIEQSFSEEHLCFRTLQRFTAATLEHGMRPPIS
PKPEWRALLDEMAVVATEEYRSVVFQEPRFVEYFRLATPELEYGRMNIGSRPSKRKPSGGIESL
RAIPWIFAWIQTRFHLPVWLGFGSAIRHVIEKDVRNLHMLQDMYQHWPFFRVTIDLIEMVFAKG
DPGIAALYDKLLVSEELWPFGEKLRANFEETKKLILQTAGHKDLLEGDPYLKQGLRLRDSYITT
LNVCQAYTLKRIRDPSYHVTLRPHISKEIAESSKPAKELIELNPTSEYAPGLEDTLILTMKGIA
AGLQNTG
SEQ ID NO: 15_(Zm_PPC1, C4, shoot, photosynthesis)
MASTKAPGPGEKHHSIDAQLRQLVPGKVSEDDKLIEYDALLVDRFLNILQDLHGPSLREF
VQECYEVSADYEGKGDTTKLGELGAKLTGLAPADAILVASSILHMLNLANLAEEVQIAHR
RRNSKLKKGGFADEGSATTESDIEETLKRLVSEVGKSPEEVFEALKNQTVDLVFTAHPTQ
SARRSLLQKNARIRNCLTQLNAKDITDDDKQELDEALQREIQAAFRTDEIRRAQPTPQDE
MRYGMSYIHETVWKGVPKFLRRVDTALKNIGINERLPYNVSLIRFSSWMGGDRDGNPRVT
PEVTRDVCLLARMMAANLYIDQIEELMFELSMWRCNDELRVRAEELHSSSGSKVTKYYIE
FWKQIPPNEPYRVILGHVRDKLYNTRERARHLLASGVSEISAESSFTSIEEFLEPLELCY
KSLCDCGDKAIADGSLLDLLRQVFTFGLSLVKLDIRQESERHTDVIDAITTHLGIGSYRE
WPEDKRQEWLLSELRGKRPLLPPDLPQTDEIADVIGAFHVLAELPPDSFGPYIISMATAP
SDVLAVELLQRECGVRQPLPVVPLFERLADLQSAPASVERLFSVDWYMDRIKGKQQVMVG
YSDSGKDAGRLSAAWQLYRAQEEMAQVAKRYGVKLTLFHGRGGTVGRGGGPTHLAILSQP
PDTINGSIRVTVQGEVIEFCFGEEHLCFQTLQRFTAATLEHGMHPPVSPKPEWRKLMDEM
AVVATEEYRSVVVKEARFVEYFRSATPETEYGRMNIGSRPAKRRPGGGITTLRAIPWIFS
WTQTRFHLPVWLGVGAAFKFAIDKDVRNFQVLKEMYNEWPFFRVTLDLLEMVFAKGDPGI
AGLYDELLVAEELKPFGKQLRDKYVETQQLLLQIAGHKDILEGDPFLKQGLVLRNPYITT
LNVFQAYTLKRIRDPNFKVTPQPPLSKEFADENKPAGLVKLNPASEYPPGLEDTLILTMK
GIAAGMQNTG
In SEQ ID NO: 15: i) S at position 780 is a hallmark for C4 and
would be A if C3; and ii) G at position 890 is a hallmark for C4
and would be R if C3.

1.-43. (canceled)

44. A plant expressing a variant phosphoenolpyruvate carboxylase (PEPC), wherein the variant PEPC comprises one or more amino acid substitutions selected from the group consisting of A651V, G678S, A776S, T778I, and R886G in the consensus sequence of SEQ ID NO: 8.

45. The plant of claim 44, wherein the variant PEPC comprises one or more amino acid substitutions selected from the group consisting of A651V, G678S, and T778I in the consensus sequence of SEQ ID NO:8.

46. The plant of claim 44, wherein the variant PEPC comprises an amino acid substitution selected from the group consisting of one or both of A776S and R886G in the consensus sequence of SEQ ID NO: 8.

47. The plant of claim 44, wherein the amino acid sequence of the variant PEPC comprises SEQ ID NO:9, and the amino acid sequence of the variant does not consist of SEQ ID NO: 10, SEQ ID NO: 11, or SEQ ID NO: 12.

48. The plant of claim 44, wherein the variant PEPC comprises one or more further amino acid substitutions selected from the group consisting of R637G, R637A, R637V, R637I, R637L, R637M, Q675G, Q675A, Q675V, Q675I, Q675L, Q675M, H675G, H675A, H675V, H675I, H675L, H675M, K831G, K831A, K831V, K831I, K831L, K831M, R890G, R890A, R890V, R890I, R890L, R890M, N965G, N965A, N965V, N965I, N965L, and N965M.

49. The plant of claim 44, wherein the variant PEPC is expressed in roots of the plant.

50. The plant of claim 49, wherein the plant has increased malate production.

51. The plant of claim 44, wherein the plant has enhanced aluminum resistance, increased glucose production derived from C4 photosynthesis, and increased oxaloacetate-derived amino acid production as compared to a control plant that does not express the variant PEPC.

52. The plant of claim 51, wherein growth of the plant is greater in a phytotoxic substrate as compared to the control plant when grown under the same conditions, and wherein the phytotoxic substrate is a growth substrate having a pH from 2-5 and nanomolar or higher levels of Al³⁺.

53. The plant of claim 44, wherein the plant is selected from the group consisting of maize, soybean, wheat, barley, rice, and sorghum.

54. A maize plant expressing a variant phosphoenolpyruvate carboxylase (PEPC), wherein the variant PEPC comprises one or more amino acid substitutions selected from the group consisting of A651V, G678S, A776S, T778I, and R886G in the consensus sequence of SEQ ID NO: 8.

55. The maize plant of claim 54, wherein the variant PEPC is a variant C3 or C4 PEPC, and wherein the variant PEPC comprises one or more amino acid substitutions selected from the group consisting of A651V, G678S, and T778I in the consensus sequence of SEQ ID NO:8.

56. The maize plant of claim 54, wherein the variant PEPC is a C3 PEPC, and wherein the variant PEPC comprises an amino acid substitution selected from the group consisting of one or both of A776S and R886G in the consensus sequence of SEQ ID NO: 8.

57. The maize plant of claim 54, wherein the amino acid sequence of the variant PEPC comprises SEQ ID NO:9, and the amino acid sequence of the variant does not consist of SEQ ID NO: 10, SEQ ID NO: 11, or SEQ ID NO: 12.

58. The maize plant of claim 54, wherein the variant PEPC comprises one or more further amino acid substitutions selected from the group consisting of R637G, R637A, R637V, R637I, R637L, R637M, Q675G, Q675A, Q675V, Q675I, Q675L, Q675M, H675G, H675A, H675V, H675I, H675L, H675M, K831G, K831A, K831V, K831I, K831L, K831M, R890G, R890A, R890V, R890I, R890L, R890M, N965G, N965A, N965V, N965I, N965L, and N965M.

59. The maize plant of claim 54, wherein the variant PEPC is expressed in roots of the plant.

60. The maize plant of claim 59, wherein the plant has increased malate production.

61. The maize plant of claim 54, wherein the plant has enhanced aluminum resistance, increased glucose production derived from C4 photosynthesis, and increased oxaloacetate-derived amino acid production as compared to a control maize plant that does not express the variant PEPC.

62. The maize plant of claim 61, wherein growth of the plant is greater in a phytotoxic substrate as compared to the control plant when grown under the same conditions, and wherein the phytotoxic substrate is a growth substrate having a pH from 2-5 and nanomolar or higher levels of Al³⁺.
